(19) United States
(12) Patent Application Publication
(10) Pub. No.: US 2016/0025730 A1
(43) Pub. Date: Jan. 28, 2016

(54) METHODS OF SELECTING COMBINATION THERAPY FOR COLORECTAL CANCER PATIENTS

(71) Applicant: NESTEC S.A., Vevey (CH)

(72) Inventors: Phillip KIM, Irvine, CA (US); Xinjun LIU, San Diego, CA (US); Sharat SINGH, Rancho Santa Fe, CA (US)

(73) Assignee: NESTEC S.A., Vevey (CH)

(21) Appl. No.: 14/815,870

(22) Filed: Jul. 31, 2015

Related U.S. Application Data
(63) Continuation of application No. PCT/IB2014/058820, filed on Feb. 5, 2014.
(60) Provisional application No. 61/761,164, filed on Feb. 5, 2013.

(51) Int. Cl. G01N 33/574

(52) U.S. Cl. CPC G01N 33/57419; G01N 2800/52

(57) ABSTRACT

The present invention provides methods for selecting a subject as suitable for combination therapy with EGFR and HER2 inhibitors. The present invention also provides methods for predicting whether a subject will benefit from the combination therapy. In some embodiments, the present invention provides methods for determining whether to administer a combination therapy in a subject receiving EGFR inhibitor therapy. In other embodiments, the present invention provides methods for monitoring a subject on EGFR inhibitor therapy to determine whether to administer a combination therapy of EGFR and HER2 inhibitors. The present invention is particularly useful to facilitate the design of personalized therapies for colorectal cancer patients with EGFR inhibitor sensitivity.

### CROSS REFERENCE TO RELATED APPLICATIONS

This application is a continuation of PCT/IB2014/058820, filed Feb. 5, 2014, which application claims priority to U.S. Provisional Application No. 61/761,164, filed Feb. 5, 2013, the disclosures of which are hereby incorporated by reference in their entirety for all purposes.

### BACKGROUND OF THE INVENTION

The process of signal transduction in cells is responsible for a variety of biological functions including, but not limited to, cell division and death, metabolism, immune cell activation, neurotransmission, and sensory perception to name but a few. Accordingly, derangements in normal signal transduction in cells can lead to a number of disease states such as diabetes, heart disease, autoimmunity, and cancer.

One well characterized signal transduction pathway is the MAP kinase pathway, which is responsible for transducing the signal from epidermal growth factor (EGF) to the promotion of cell proliferation in cells. EGF binds to a transmembrane receptor-linked tyrosine kinase, the epidermal growth factor receptor (EGFR), which is activated by the binding of EGF. The binding of EGF to EGFR activates the tyrosine kinase activity of the cytoplasmic domain of the receptor. One consequence of this kinase activation is the autophosphorylation of EGFR on tyrosine residues. The phosphorylated tyrosine residues on the activated EGFR provide a docking site for the binding of SH2 domain containing adaptor proteins such as GRB2. In its function as an adaptor, GRB2 further binds to a guanine nucleotide exchange factor, SOS, by way of an SH3 domain on GRB2. The formation of the complex of EGFR-GRB2-SOS leads to SOS activation to a guanine nucleotide exchange factor that promotes the removal of GDP from Ras. Upon removal of GDP, Ras binds GTP and becomes activated.

Following activation, Ras binds to and activates the protein kinase activity of RAF kinase, a serine/threonine-specific protein kinase. What follows is the activation of a protein kinase cascade that leads to cell proliferation. In outline, RAF kinase then phosphorylates and activates MEK, another serine/threonine kinase. Activated MEK phosphorylates and activates mitogen-activated protein kinase (MAPK). Among the targets for further phosphorylation by MAPK are 40S ribosomal protein S6 kinase (RSK). The phosphorylation of RSK by MAPK results in activation of RSK, which in turn phosphorylates ribosomal protein S6. Another known target of MAPK is the proto-oncogene, c-Myc, a gene important for cell proliferation, which is mutated in a variety of cancers. MAPK also phosphorylates and activates another protein kinase, MNK, which in turn phosphorylates the transcription factor, CREB. Indirectly, MAPK also regulates the transcription of the Fos gene, which encodes yet another transcription factor involved in cell proliferation. By altering the levels and activities of such transcription factors, MAPK transduces the original extracellular signal from EGF into altered transcription of genes that are important for cell cycle progression.

Given the central role that signal transduction pathways play in cell growth, it is not surprising that many cancers arise as a result of mutations and other alterations in signal transduction components that result in aberrant activation of cell proliferation pathways. For example, overexpression or hyperactivity of EGFR has been associated with a number of cancers, including glioblastoma multiforme, colon cancer, and lung cancer. This has prompted the development of anticancer therapeutics directed against EGFR, including gefitinib and erlotinib for lung cancer, and cetuximab for colon cancer.

Cetuximab is an example of a monoclonal antibody inhibitor, which binds to the extracellular ligand binding domain of EGFR, thus preventing the binding of ligands which activate the EGFR tyrosine kinase. In contrast, gefitinib and erlotinib are small molecules which inhibit the intracellularly-located EGFR tyrosine kinase. In the absence of kinase activity, EGFR is unable to undergo autophosphorylation at tyrosine residues, which is a prerequisite for binding of downstream adaptor proteins, such as GRB2. By halting the signaling cascade in cells that rely on this pathway for growth, tumor proliferation and migration is diminished.

Additionally, other studies have shown that about 70% of human melanomas and a smaller fraction of other tumors have a point mutation (V599E) in the Raf gene which leads to persistent activation of the MAPK pathway (see, e.g., Davies et al., Nature, 417:949-954 (2002)). Such results suggest that mutations in particular signal transduction pathways may be characteristic of particular types of tumors and that such specific, altered signal transduction pathways may be a promising target for chemotherapeutic intervention.

Given that different cancer treatments, particularly cancer chemotherapy, may function either directly or indirectly by means of either blocking or activating cellular signal transduction pathways involved in cell proliferation or death, respectively, the expression and/or activation of a given signal transduction pathway in a particular form of cancer such as, for example, colorectal cancer may serve as a good indicator of the efficacy of various cancer treatments. Accordingly, in addition to fulfilling other needs, the present invention provides a method for evaluating the effectiveness of potential anticancer therapies for an individual patient with colorectal cancer. As such, the present invention provides methods for assisting a physician in selecting a suitable cancer therapy for the treatment of colorectal cancer at the right dose and at the right time for every patient.

### BRIEF SUMMARY OF THE INVENTION

In certain aspects, the present invention provides methods for selecting a subject as suitable for combination therapy with both an EGFR (ErbB1) inhibitor and a HER2 inhibitor. In other aspects, the present invention provides methods for predicting whether a subject will benefit from combination therapy. In particular embodiments of the invention, methods are provided for determining whether to administer a combination therapy in a subject receiving therapy with an EGFR inhibitor. In further aspects, the present invention provides methods for monitoring a subject receiving therapy with an EGFR inhibitor to determine whether to administer a combination therapy comprising the EGFR inhibitor with a HER2 inhibitor.

In particular aspects, the present invention provides methods for therapy selection, prediction, and monitoring by detecting and/or quantifying the expression (e.g., total) levels and/or activation levels of one or a plurality of dysregulated signal transduction molecules in tumor tissue including complexes thereof such as ErbB dimers (e.g., heterodimers of HER2 and HER3) and/or HER3:PI3K complexes. In certain embodiments, the expression and/or activation levels of molecular complexes such as ErbB dimers (e.g., heterodimers of HER2 and HER3) and HER3:PI3K complexes are detected and/or quantified with an immunoassay, e.g., a specific, multiplex, high-throughput assay, such as a Collaborative Enzyme Enhanced Reactive Immunoassay (CEER). Thus, the present invention can advantageously be used to facilitate the design of personalized therapies for EGFR inhibitor-sensitive patients such as colorectal cancer patients receiving EGFR inhibitor therapy.

In one aspect, the present invention provides a method for determining whether to administer combination therapy in a subject receiving therapy with an EGFR inhibitor, the method comprising:
(a) detecting and/or quantifying the level of a complex in a sample taken from the subject, wherein the complex comprises an ErbB dimer, a HER3:PI3K complex, or a combination thereof; and
(b) determining whether to administer a combination therapy comprising an EGFR inhibitor and a HER2 inhibitor based upon the level of the complex in the sample.

In some embodiments, the subject is sensitive to an EGFR inhibitor such as, e.g., cetuximab. In other embodiments, the ErbB dimer is an ErbB receptor heterodimer such as, e.g., a HER2:HER3 heterodimer. In certain embodiments, the subject should be administered the combination therapy when the level of the ErbB dimer or the HER3:PI3K complex that is detected and/or quantified in the subject's sample is higher than a reference level thereof. In particular embodiments, the subject should be administered the combination therapy when the levels of both the ErbB dimer and the HER3:PI3K complex detected and/or quantified in the subject's sample are higher than the reference levels thereof. In yet other embodiments, the method further comprises detecting and/or quantifying the expression and/or activation level of HER2 and/or HER3 in the sample. In particular embodiments, the administration of the combination therapy reduces and/or inhibits the formation of the ErbB dimer and/or the HER3:PI3K complex. In some embodiments, the combination therapy also reduces and/or inhibits HER2 expression, HER3 expression, and/or HER3 activation (e.g., phosphorylation). Without wishing to be bound by any particular theory, the present inventors have discovered that combination therapy with EGFR and HER2 inhibitors increases the therapeutic index in EGFR inhibitor-sensitive subjects (e.g., cetuximab-sensitive patients) due to the inhibition or suppression of feedback mechanisms that are activated or induced upon EGFR inhibition.

In another aspect, the present invention provides a method for monitoring a subject receiving therapy with an EGFR inhibitor, the method comprising:
(a) detecting and/or quantifying the level of a complex in a sample taken from the subject at time (t₂), wherein the complex comprises an ErbB dimer, a HER3:PI3K complex, or a combination thereof; and
(b) comparing the level of the complex detected and/or quantified at (t₂) to the level of the complex detected and/or quantified at an earlier time (t₁); and
(c) determining whether to administer a combination therapy comprising the EGFR inhibitor with a HER2 inhibitor based upon a difference between the level of the complex at (t₂) compared to (t₁).

In some embodiments, the subject is sensitive to an EGFR inhibitor such as, e.g., cetuximab. In other embodiments, the ErbB dimer is an ErbB receptor heterodimer such as, e.g., a HER2:HER3 heterodimer. In some embodiments, the subject should be administered the combination therapy when the level of the ErbB dimer or the HER3:PI3K complex that is detected and/or quantified in the subject's sample is higher at (t₂) compared to (t₁). In other embodiments, the subject should be administered the combination therapy when the levels of both the ErbB dimer and the HER3:PI3K complex detected and/or quantified in the subject's sample are higher at (t₂) compared to (t₁). In certain other embodiments, the method further comprises detecting and/or quantifying the expression and/or activation level of HER2 and/or HER3 in the sample. In particular embodiments, the administration of the combination therapy reduces and/or inhibits the formation of the ErbB dimer and/or the HER3:PI3K complex. In some embodiments, the combination therapy also reduces and/or inhibits HER2 expression, HER3 expression, and/or HER3 activation (e.g., phosphorylation). Without wishing to be bound by any particular theory, the present inventors have discovered that EGFR inhibitor-sensitive subjects (e.g., cetuximab-sensitive patients) on EGFR inhibitor therapy can be monitored for the administration of combination therapy with EGFR and HER2 inhibitors to increase the therapeutic index due to the inhibition or suppression of feedback mechanisms that are activated or induced upon EGFR inhibition.

In sum, the methods of the present invention provide accurate prediction, selection, and monitoring of EGFR inhibitor-sensitive patients, such as, e.g., colorectal cancer patients receiving EGFR inhibitor therapy, most likely to benefit from targeted combination therapy by performing pathway profiling on signal transduction molecules (e.g., complexes of ErbB receptors and/or PI3K protein complexes) in patient tumor tissue samples and determining whether to administer a combination therapy comprising an EGFR inhibitor together with a HER2 inhibitor based upon the level of expression and/or activation of these molecules or complexes thereof.

Other objects, features, and advantages of the present invention will be apparent to one of skill in the art from the following detailed description and figures.

### BRIEF DESCRIPTION OF THE DRAWINGS

FIG. 1 shows the expression and/or activation levels of HER1, HER2, HER3, AKT, ERK, MEK, and RSK in Lim1215 cells during the course of treatment with cetuximab.

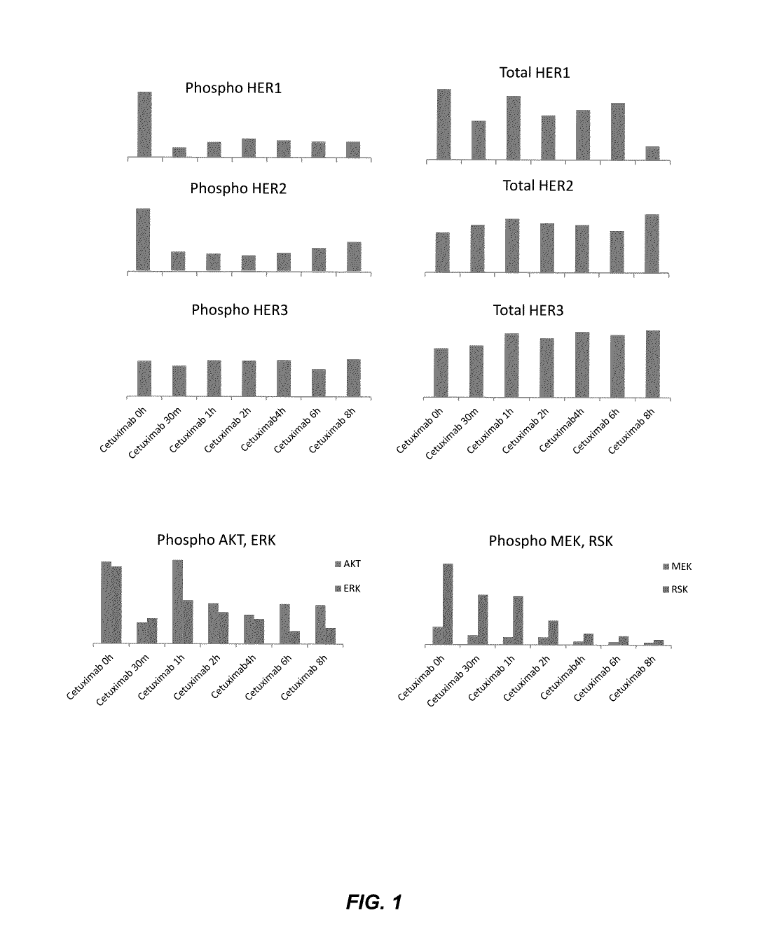

FIG. 2 shows the expression and/or activation levels of HER1, HER2, HER3, AKT, ERK, MEK, and RSK in Lim1215 cells during the course of treatment with cetuximab, pertuzumab, trastuzumab, a HER3 inhibitor, and combinations thereof.

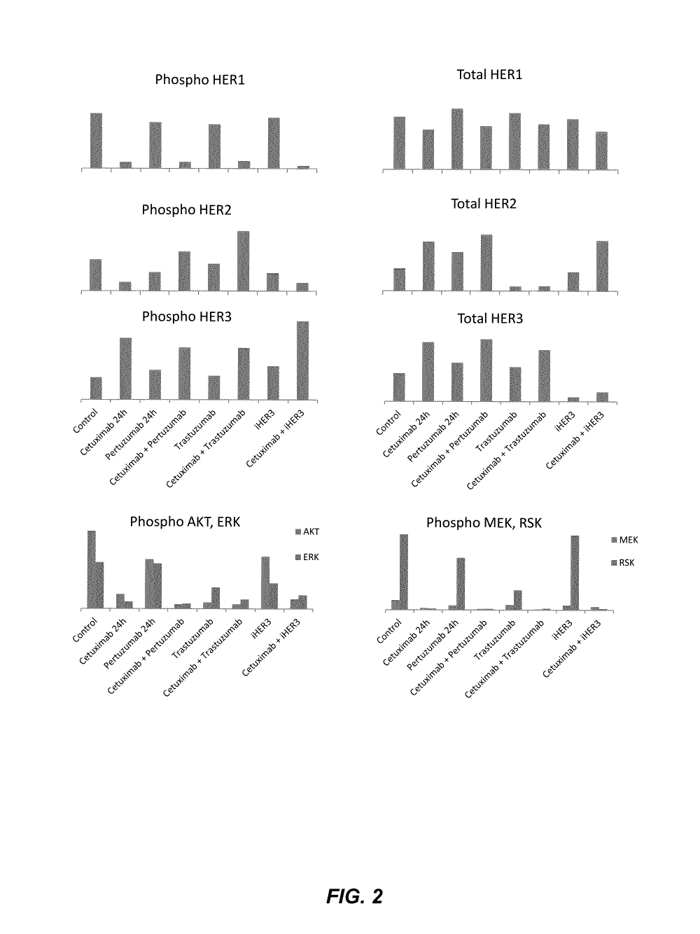

FIG. 3 shows the expression and/or activation levels of HER1, HER2, HER3, AKT, ERK, MEK, and RSK in Lim1215 cells treated with cetuximab, pertuzumab, trastuzumab, and combinations thereof, as compared to cells treated with gefitinib and lapatinib.

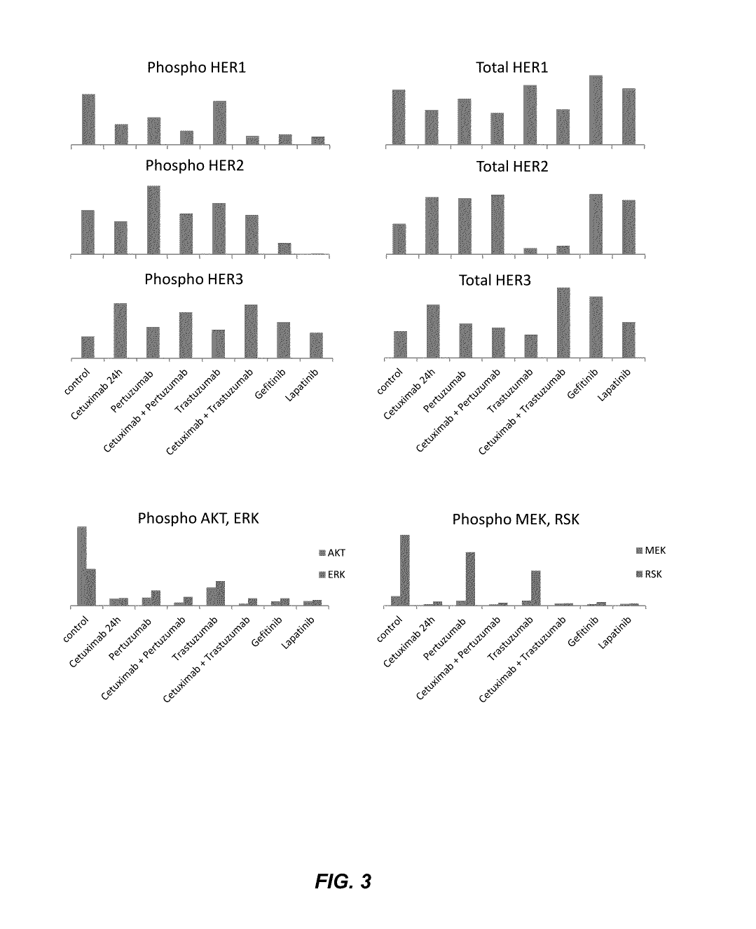

FIG. 4 shows the expression and/or activation levels of HER1, HER2, HER3, AKT, ERK, MEK, and RSK in Lim1215 cells treated with cetuximab, gefitinib, lapatinib, and MEK inhibitor AS703026.

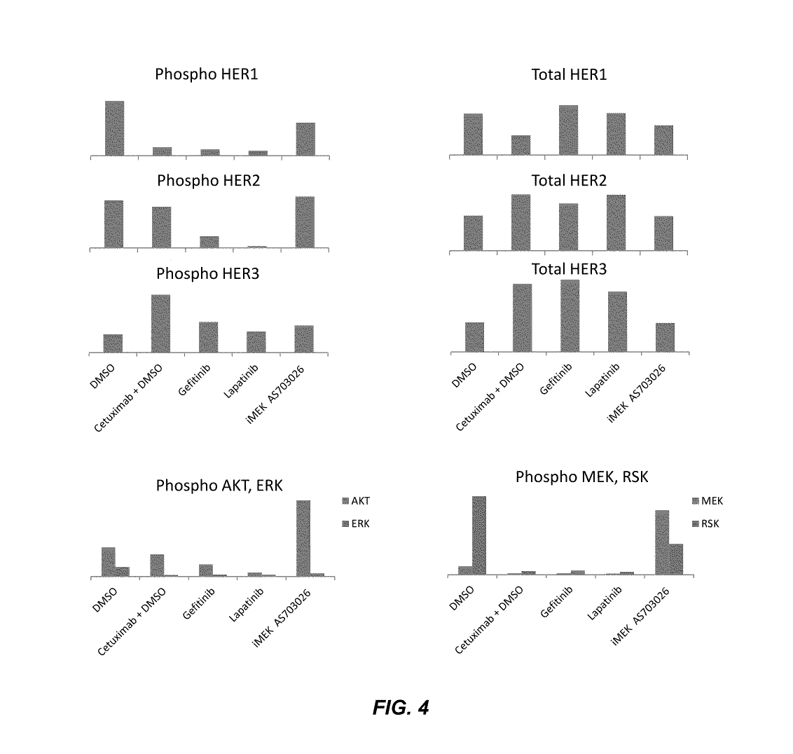

FIGS. 5A, 5B, and 5D show the expression and/or activation levels of HER1, HER2, HER3, AKT, ERK, MEK, and RSK in Lim1215 cells during the course of treatment with cetuximab. FIG. 5C shows the level of HER heterodimers and HER3:PI3K dimers during the course of treatment with cetuximab.

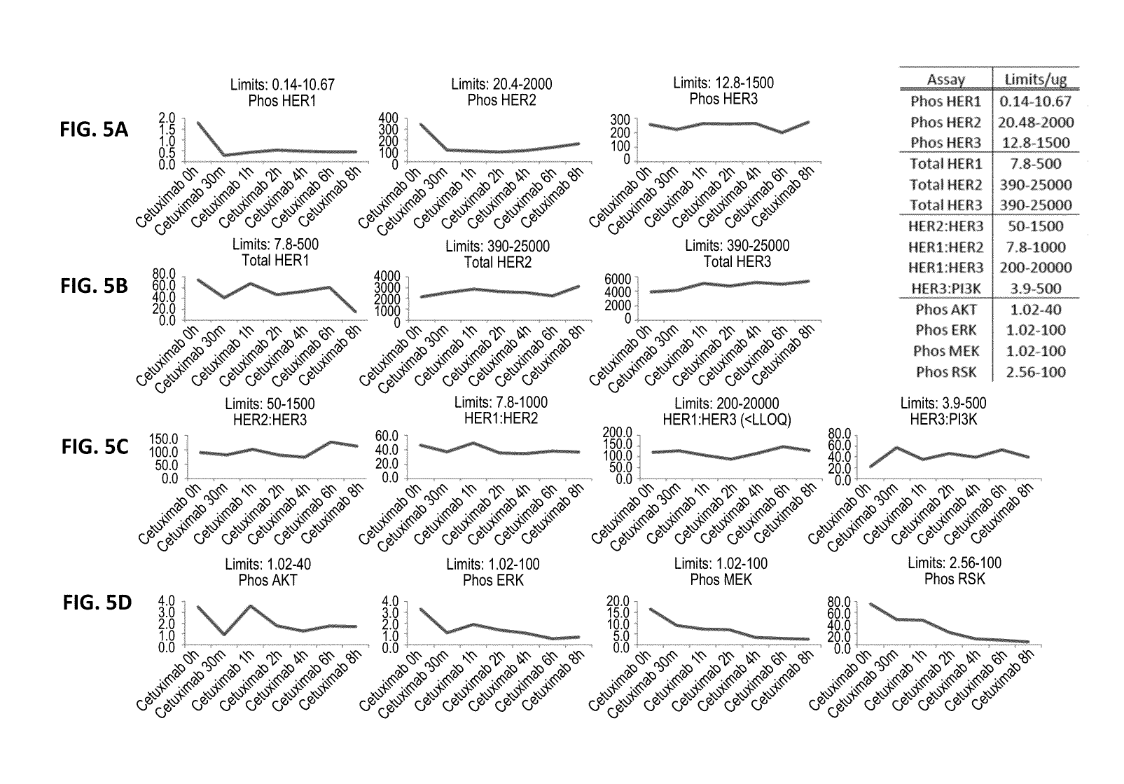

FIGS. 6A, 6B, 6D, and 6E show the expression and/or activation levels of HER1, HER2, HER3, AKT, ERK, MEK, and RSK in Lim1215 cells treated with cetuximab for 24 hours. FIGS. 6C and 6F show the level of HER heterodimers and HER3:PI3K dimers treated with cetuximab for 24 hours.

FIGS. 7A, 7B, 7D, and 7E show the expression and/or activation levels of HER1, HER2, HER3, AKT, ERK, MEK, and RSK in Lim1215 cells treated with pertuzumab for 24 hours. FIGS. 7C and 7F show the level of HER heterodimers and HER3:PI3K dimers treated with pertuzumab for 24 hours.

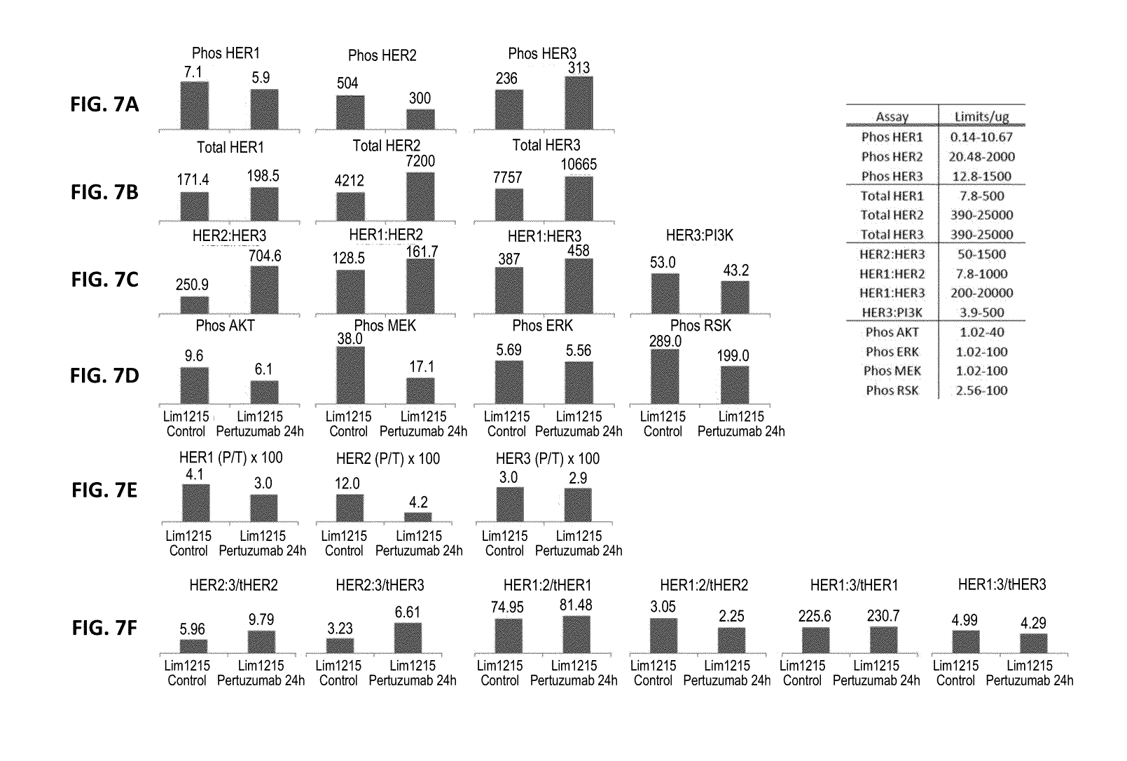

FIGS. 8A, 8B, 8D, and 8E show the expression and/or activation levels of HER1, HER2, HER3, AKT, ERK, MEK, and RSK in Lim1215 cells treated with trastuzumab for 24 hours. FIGS. 8C and 8F show the level of HER heterodimers and HER3:PI3K dimers treated with trastuzumab for 24 hours.

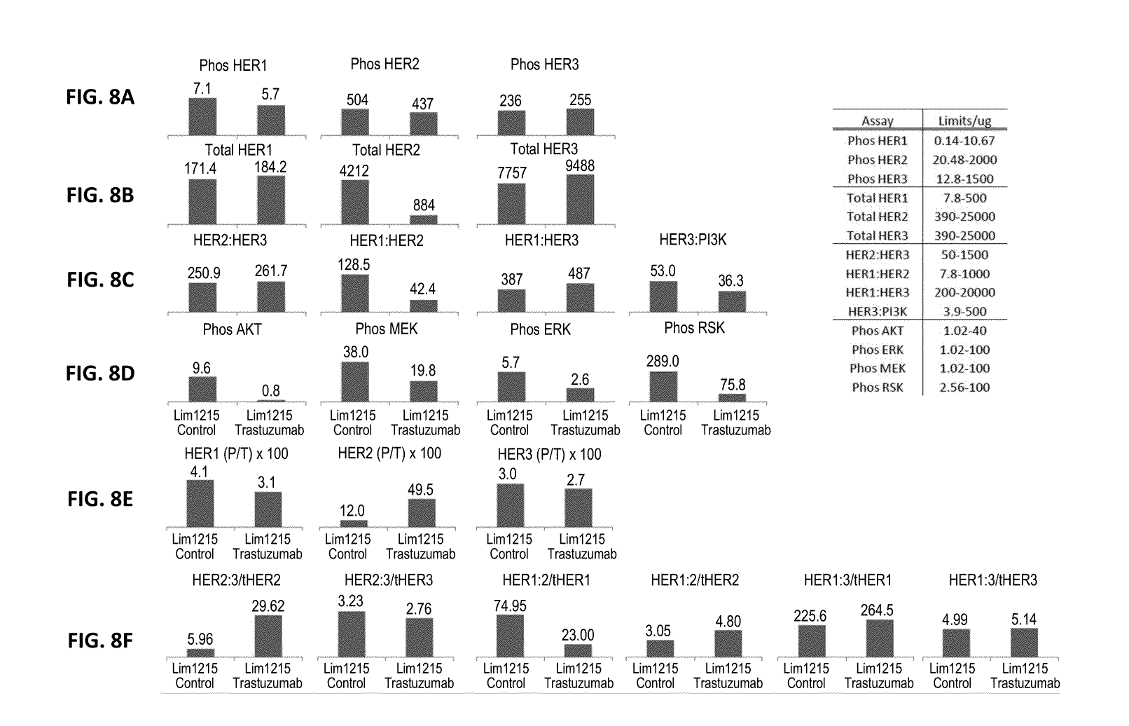

FIGS. 9A, 9B, 9D, and 9E show the expression and/or activation levels of HER1, HER2, HER3, AKT, ERK, MEK, and RSK in Lim1215 cells treated with a HER3 inhibitor for 24 hours. FIGS. 9C and 9F show the level of HER heterodimers and HER3:PI3K dimers treated with a HER3 inhibitor for 24 hours.

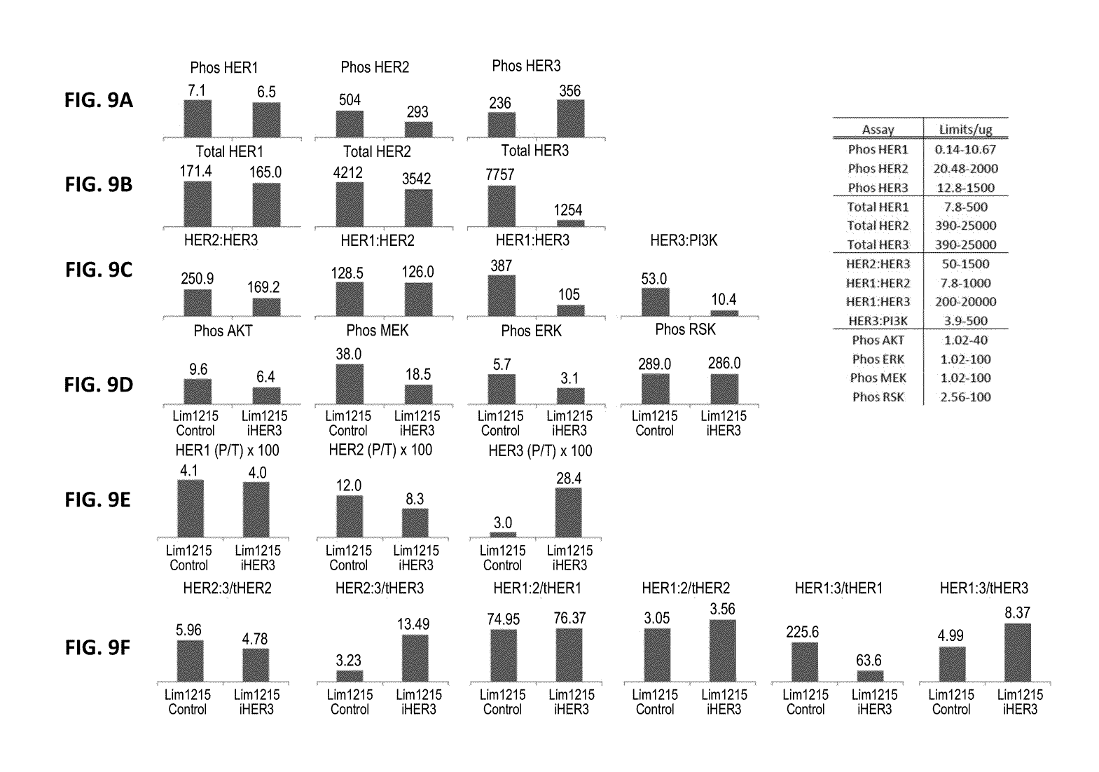

FIGS. 10A, 10B, 10D, and 10E show the expression and/or activation levels of HER1, HER2, HER3, AKT, ERK, MEK, and RSK in Lim1215 cells treated with cetuximab and pertuzumab for 24 hours. FIG. 10C shows the level of HER heterodimers and HER3:PI3K dimers treated with cetuximab and pertuzumab for 24 hours.

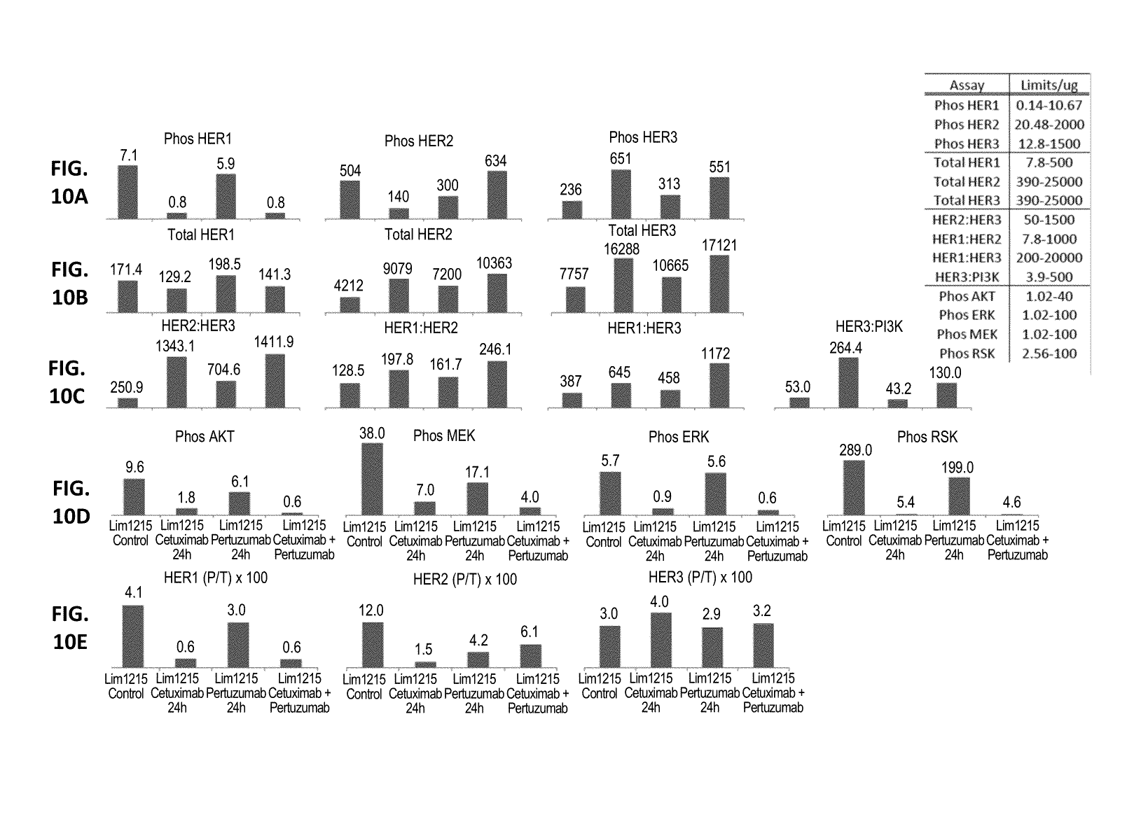

FIGS. 11A, 11B, 11D, and 11E show the expression and/or activation levels of HER1, HER2, HER3, AKT, ERK, MEK, and RSK in Lim1215 cells treated with cetuximab and trastuzumab for 24 hours. FIG. 11C shows the level of HER heterodimers and HER3:PI3K dimers treated with cetuximab and trastuzumab for 24 hours.

FIGS. 12A, 12B, 12D, and 12E show the expression and/or activation levels of HER1, HER2, HER3, AKT, ERK, MEK, and RSK in Lim1215 cells treated with cetuximab and a HER3 inhibitor for 24 hours. FIG. 12C shows the level of HER heterodimers and HER3:PI3K dimers treated with cetuximab and a HER3 inhibitor for 24 hours.

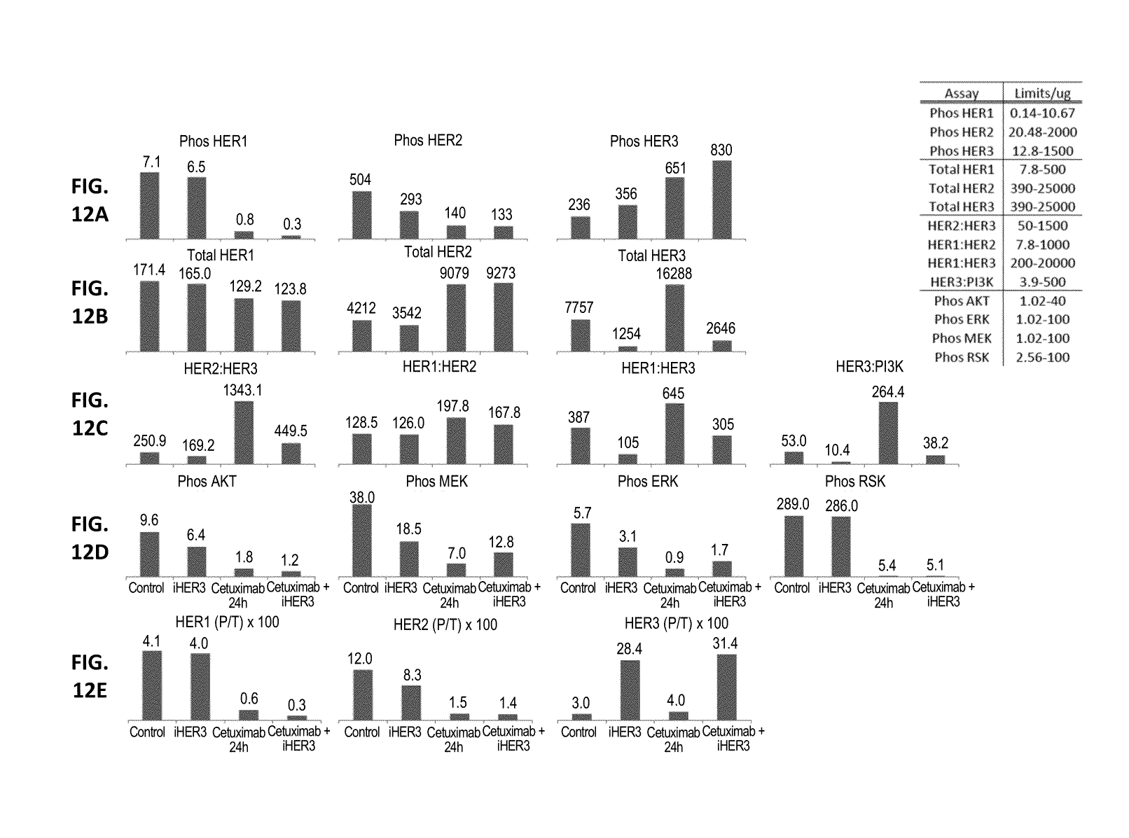

FIGS. 13A, 13B, and 13D show the expression and/or activation levels of HER1, HER2, HER3, AKT, ERK, MEK, and RSK in Lim1215 cells treated with cetuximab, pertuzumab, trastuzumab, a HER3 inhibitor, and combinations thereof for 24 hours. FIG. 13C shows the level of HER heterodimers and HER3:PI3K dimers treated with cetuximab, pertuzumab, trastuzumab, a HER3 inhibitor, and combinations thereof for 24 hours.

FIGS. 14A, 14B, and 14D show the expression and/or activation levels of HER1, HER2, HER3, AKT, ERK, MEK, and RSK in Lim1215 cells treated with cetuximab for 24 hours. FIG. 14C shows the level of HER heterodimers and HER3:PI3K dimers treated with cetuximab for 24 hours.

FIGS. 15A, 15B, and 15D show the expression and/or activation levels of HER1, HER2, HER3, AKT, ERK, MEK, and RSK in Lim1215 cells treated with gefitinib for 24 hours. FIG. 15C shows the level of HER heterodimers and HER3:PI3K dimers treated with gefitinib for 24 hours.

FIGS. 16A, 16B, and 16D show the expression and/or activation levels of HER1, HER2, HER3, AKT, ERK, MEK, and RSK in Lim1215 cells treated with lapatinib for 24 hours. FIG. 16C shows the level of HER heterodimers and HER3:PI3K dimers treated with lapatinib for 24 hours.

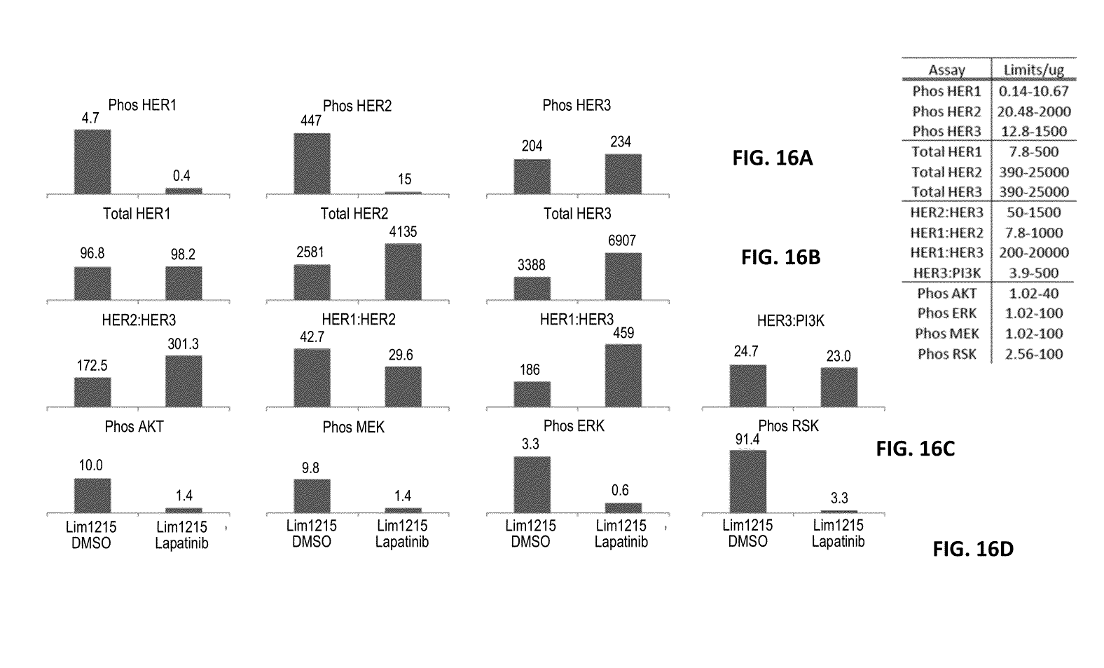

FIGS. 17A, 17B, and 17D show the expression and/or activation levels of HER1, HER2, HER3, AKT, ERK, MEK, and RSK in Lim1215 cells treated with an MEK inhibitor for 24 hours. FIG. 17C shows the level of HER heterodimers and HER3:PI3K dimers treated with an MEK inhibitor for 24 hours.

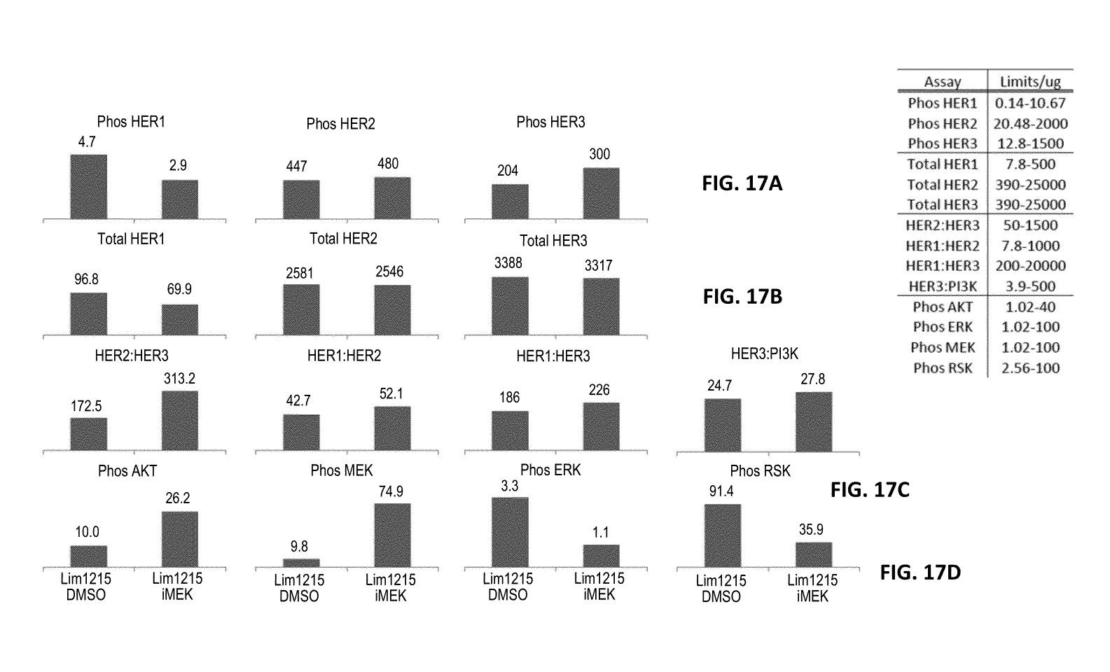

FIGS. 18A, 18B, and 18D show the expression and/or activation levels of HER1, HER2, HER3, AKT, ERK, MEK, and RSK in Lim1215 cells treated with cetuximab, gefitinib, lapatinib, or an MEK inhibitor for 24 hours. FIG. 18C shows the level of HER heterodimers and HER3:PI3K dimers treated with cetuximab, gefitinib, lapatinib, or an MEK inhibitor for 24 hours.

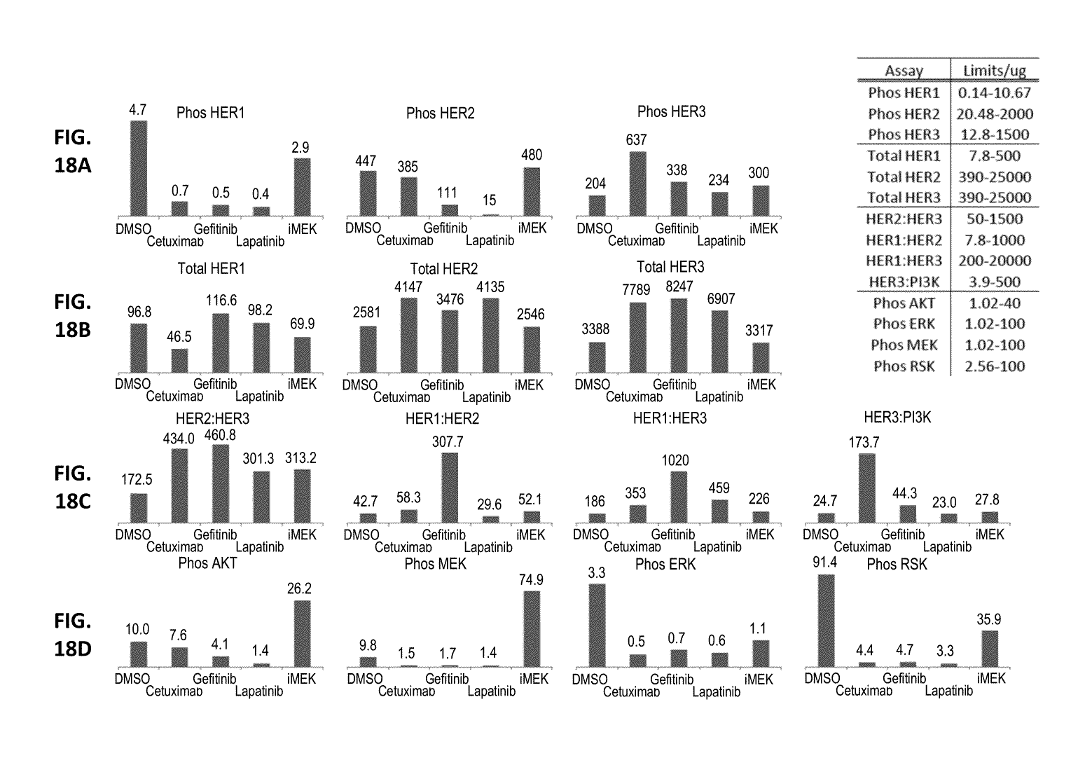

### DETAILED DESCRIPTION OF THE INVENTION

### I. Introduction

The present invention provides methods for selecting, identifying, and monitoring a subject on EGFR inhibitor therapy (e.g., an EGFR inhibitor-sensitive subject) as suitable for combination therapy with both an EGFR inhibitor and a HER2 inhibitor for the treatment of a cancer such as colorectal cancer. The present invention is based, in part, upon the surprising discovery that signal transduction pathway profiling of cancer cells using an immunoassay such as a Collaborative Enzyme Enhanced Reactive Immunoassay (CEER) advantageously provides critical information for selecting the most effective targeted therapeutic agents for combination therapy to increase the therapeutic index for treating a cancer such as colorectal cancer, e.g., when compared to monotherapy with an EGFR inhibitor alone. Therefore, the present invention can be used to facilitate the design of personalized therapies for subjects sensitive to EGFR inhibitors (e.g., colorectal cancer subjects on EGFR inhibitor therapy).

Example 1 below demonstrates that co-treatment of EGFR inhibitor-sensitive colorectal cancer cells (e.g., a cetuximab-sensitive human colon cancer cell line such as Lim1215 cells) with a combination of EGFR and HER2 inhibitors relieves or rescues the feedback mechanisms that are activated or induced when the cells are treated with EGFR inhibitor alone. As illustrated in Example 1, feedback mechanisms that are activated or induced when Lim1215 cells are treated with EGFR inhibitor alone, but are inhibited or suppressed when Lim1215 cells are co-treated with a combination of EGFR and HER2 inhibitors include, without limitation, ErbB receptor dimer formation (e.g., HER2:HER3 heterodimer formation), HER3:PI3K complex formation, increased expression of HER2, increased expression of HER3, increased HER3 phosphorylation level, and combinations thereof. These results are predictive of and support methods for combination therapy with EGFR and HER2 inhibitors to increase the therapeutic index in EGFR inhibitor-sensitive subjects by inhibiting or suppressing feedback signaling mechanisms that are activated or induced when only EGFR is inhibited.

### II. Definitions

An “inhibitor” includes an agent (e.g., a compound, molecule, etc.) that binds to an analyte such as a polypeptide and inhibits, partially or totally blocks stimulation or enzymatic activity, decreases, prevents, delays activation, inactivates, desensitizes, or down-regulates the activity of the analyte.

The term “analyte” includes any molecule of interest, typically a macromolecule such as a polypeptide, whose presence, amount (expression level), activation state or level, and/or identity is determined.

The term “signal transduction molecule” or “signal transducer” includes proteins and other molecules that carry out the process by which a cell converts an extracellular signal or stimulus into a response, typically involving ordered sequences of biochemical reactions inside the cell. Examples of signal transduction molecules include, but are not limited to, receptor tyrosine kinases such as EGFR (e.g., EGFR/HER1/ErbB1, HER2/Neu/ErbB2, HER3/ErbB3, HER4/ErbB4), VEGFR1/FLT1, VEGFR2/FLK1/KDR, VEGFR3/FLT4, FLT3/FLK2, PDGFR (e.g., PDGFRA, PDGFRB), c-KIT/SCFR, INSR (insulin receptor), IGF-IR, IGF-IIR, IRR (insulin receptor-related receptor), CSF-1R, FGFR 1-4, HGFR 1-2, CCK4, TRK A-C, c-MET, RON, EPHA 1-8, EPHB 1-6, AXL, MER, TYRO3, TIE 1-2, TEK, RYK, DDR 1-2, RET, c-ROS, V-cadherin, LTK (leukocyte tyrosine kinase), ALK (anaplastic lymphoma kinase), ROR 1-2, MUSK, AATYK 1-3, and RTK 106; truncated forms of receptor tyrosine kinases such as truncated HER2 receptors with missing amino-terminal extracellular domains (e.g., p95ErbB2 (p95m), p110, p95c, p95n, etc.), truncated cMET receptors with missing amino-terminal extracellular domains, and truncated HER3 receptors with missing amino-terminal extracellular domains; receptor tyrosine kinase dimers (e.g., p95HER2:HER3; p95HER2:HER2; truncated HER3 receptor with HER1, HER2, HER3, or HER4; HER2:HER2; HER3:HER3; HER2:HER3; HER1:HER2; HER1:HER3; HER2:HER4; HER3:HER4; etc.); non-receptor tyrosine kinases such as BCR-ABL, Src, Frk, Btk, Csk, Abl, Zap70, Fes/Fps, Fak, Jak, Ack, and LIMK; tyrosine kinase signaling cascade components such as AKT (e.g., AKT1, AKT2, AKT3), MEK (MAP2K1), ERK2 (MAPK1), ERK1 (MAPK3), PI3K (e.g., PIK3CA (p110), PIK3R1 (p85)), PDK1, PDK2, phosphatase and tensin homolog (PTEN), SGK3, 4E-BP1, P70S6K (e.g., p70 S6 kinase splice variant alpha I), protein tyrosine phosphatases (e.g., PTP1B, PTPN13, BDP1, etc.), RAF, PLA2, MEKK, JNKK, JNK, p38, Shc (p66), Ras (e.g., K-Ras, N-Ras, H-Ras), Rho, Rac1, Cdc42, PLC, PKC, p53, cyclin D1, STAT1, STAT3, phosphatidylinositol 4,5-bisphosphate (PIP2), phosphatidylinositol 3,4,5-trisphosphate (PIP3), mTOR, BAD, p21, p27, ROCK, IP3, TSP-1, NOS, GSK-3β, RSK 1-3, JNK, c-Jun, Rb, CREB, Ki67, and paxillin; nuclear hormone receptors such as estrogen receptor (ER), progesterone receptor (PR), androgen receptor, glucocorticoid receptor, mineralocorticoid receptor, vitamin A receptor, vitamin D receptor, retinoid receptor, thyroid hormone receptor, and orphan receptors; nuclear receptor coactivators and repressors such as amplified in breast cancer-1 (AIB1) and nuclear receptor corepressor 1 (NCOR), respectively; and combinations thereof.

The term “activation state” refers to whether a particular signal transduction molecule is activated. Similarly, the term “activation level” refers to what extent a particular signal transduction molecule is activated. The activation state typically corresponds to the phosphorylation, ubiquitination, and/or complexation status of one or more signal transduction molecules. Non-limiting examples of activation states (listed in parentheses) include: HER1/EGFR (EGFRvIII, phosphorylated (p-) EGFR, EGFR:Shc, ubiquitinated (u-) EGFR, p-EGFRvIII); ErbB2 (p-ErbB2, p95HER2 (truncated ErbB2), p-p95HER2, ErbB2:Shc, ErbB2:PI3K, ErbB2:EGFR, ErbB2:ErbB3, ErbB2:ErbB4); ErbB3 (p-ErbB3, truncated ErbB3, ErbB3:PI3K, p-ErbB3:PI3K, ErbB3:Shc); ErbB4 (p-ErbB4, ErbB4:Shc); c-MET (p-c-MET, truncated c-MET, c-Met:HGF complex); AKT1 (p-AKT1); AKT2 (p-AKT2); AKT3 (p-AKT3); PTEN (p-PTEN); P70S6K (p-P70S6K); MEK (p-MEK); ERK1 (p-ERK1); ERK2 (p-ERK2); PDK1 (p-PDK1); PDK2 (p-PDK2); SGK3 (p-SGK3); 4E-BP1 (p-4E-BP1); PIK3R1 (p-PIK3R1); c-KIT (p-c-KIT); ER (p-ER); IGF-1R (p-IGF-1R, IGF-1R:IRS, IRS:PI3K, p-IRS, IGF-1R:PI3K); INSR (p-INSR); FLT3 (p-FLT3); HGFR1 (p-HGFR1); HGFR2 (p-HGFR2); RET (p-RET); PDGFRA (p-PDGFRA); PDGFRB (p-PDGFRB); VEGFR1 (p-VEGFR1, VEGFR1:PLCγ, VEGFR1:Src); VEGFR2 (p-VEGFR2, VEGFR2:PLCγ, VEGFR2:Src, VEGFR2:heparin sulphate, VEGFR2:VE-cadherin); VEGFR3 (p-VEGFR3); FGFR1 (p-FGFR1); FGFR2 (p-FGFR2); FGFR3 (p-FGFR3); FGFR4 (p-FGFR4); TIE1(p-TIE1); TIE2 (p-TIE2); EPHA (p-EPHA); EPHB (p-EPHB); GSK-3β (p-GSK-3β); NFKB (p-NFKB), IKB (p-IKB, p-P65:IKB); BAD (p-BAD, BAD:14-3-3); mTOR (p-mTOR); Rsk-1 (p-Rsk-1); Jnk (p-Jnk); P38 (p-P38); STAT1 (p-STAT1); STAT3 (p-STAT3); FAK (p-FAK); RB (p-RB); Ki67; p53 (p-p53); CREB (p-CREB); c-Jun (p-c-Jun); c-Src (p-c-Src); paxillin (p-paxillin); GRB2 (p-GRB2), Shc (p-Shc), Ras (p-Ras), GAB1 (p-GAB1), SHP2 (p-SHP2), GRB2 (p-GRB2), CRKL (p-CRKL), PLCγ (p-PLCγ), PKC (e.g., p-PKCα, p-PKCβ, p-PKCδ), adducin (p-adducin), RB1 (p-RB1), and PYK2 (p-PYK2).

As used herein, the term “dilution series” is intended to include a series of descending concentrations of a particular sample (e.g., cell lysate) or reagent (e.g., antibody). A dilution series is typically produced by a process of mixing a measured amount of a starting concentration of a sample or reagent with a diluent (e.g., dilution buffer) to create a lower concentration of the sample or reagent, and repeating the process enough times to obtain the desired number of serial dilutions. The sample or reagent can be serially diluted at least 2, 3, 4, 5, 6, 7, 8, 9, 10, 15, 20, 25, 30, 35, 40, 45, 50, 100, 500, or 1000-fold to produce a dilution series comprising at least 2, 3, 4, 5, 6, 7, 8, 9, 10, 11, 12, 13, 14, 15, 16, 17, 18, 19, 20, 25, 30, 35, 40, 45, or 50 descending concentrations of the sample or reagent. For example, a dilution series comprising a 2-fold serial dilution of a capture antibody reagent at a 1 mg/ml starting concentration can be produced by mixing an amount of the starting concentration of capture antibody with an equal amount of a dilution buffer to create a 0.5 mg/ml concentration of the capture antibody, and repeating the process to obtain capture antibody concentrations of 0.25 mg/ml, 0.125 mg/ml, 0.0625 mg/ml, 0.0325 mg/ml, etc.

The term “superior dynamic range” as used herein refers to the ability of an assay to detect a specific analyte in as few as one cell or in as many as thousands of cells. For example, the immunoassays described herein possess superior dynamic range because they advantageously detect a particular signal transduction molecule of interest in about 1-10,000 cells (e.g., about 1, 5, 10, 25, 50, 75, 100, 250, 500, 750, 1000, 2500, 5000, 7500, or 10,000 cells) using a dilution series of capture antibody concentrations.

The term “sample” as used herein includes any biological specimen obtained from a patient. Samples include, without limitation, whole blood, plasma, serum, red blood cells, white blood cells (e.g., peripheral blood mononuclear cells), ductal lavage fluid, ascites, pleural efflux, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample (e.g., tumor tissue) such as a biopsy of a tumor (e.g., needle biopsy) or a lymph node (e.g., sentinel lymph node biopsy), a tissue sample (e.g., tumor tissue) such as a surgical resection of a tumor, and cellular extracts thereof. In some embodiments, the sample is whole blood or a fractional component thereof such as plasma, serum, or a cell pellet. In other embodiments, the sample is obtained by isolating circulating cells of a solid tumor from whole blood or a cellular fraction thereof using any technique known in the art. In yet other embodiments, the sample is a formalin fixed paraffin embedded (FFPE) tumor tissue sample, e.g., from a solid tumor such as colorectal cancer. In particular embodiments, the sample is a tumor lysate or extract prepared from frozen tissue obtained from a subject having colorectal cancer.

The term “subject” or “patient” or “individual” typically includes humans, but can also include other animals such as, e.g., other primates, rodents, canines, felines, equines, ovines, porcines, and the like.

An “array” or “microarray” comprises a distinct set and/or dilution series of capture antibodies immobilized or restrained on a solid support such as, for example, glass (e.g., a glass slide), plastic, chips, pins, filters, beads (e.g., magnetic beads, polystyrene beads, etc.), paper, membrane (e.g., nylon, nitrocellulose, polyvinylidene fluoride (PVDF), etc.), fiber bundles, or any other suitable substrate. The capture antibodies are generally immobilized or restrained on the solid support via covalent or noncovalent interactions (e.g., ionic bonds, hydrophobic interactions, hydrogen bonds, Van der Waals forces, dipole-dipole bonds). In certain instances, the capture antibodies comprise capture tags which interact with capture agents bound to the solid support. The arrays used in the assays described herein typically comprise a plurality of different capture antibodies and/or capture antibody concentrations that are coupled to the surface of a solid support in different known/addressable locations.

The term “capture antibody” is intended to include an immobilized antibody which is specific for (i.e., binds, is bound by, or forms a complex with) one or more analytes of interest in a sample such as a cellular extract. In particular embodiments, the capture antibody is restrained on a solid support in an array. Suitable capture antibodies for immobilizing any of a variety of signal transduction molecules on a solid support are available from Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.).

The term “detection antibody” as used herein includes an antibody comprising a detectable label which is specific for (i.e., binds, is bound by, or forms a complex with) one or more analytes of interest in a sample. The term also encompasses an antibody which is specific for one or more analytes of interest, wherein the antibody can be bound by another species that comprises a detectable label. Examples of detectable labels include, but are not limited to, biotin/streptavidin labels, nucleic acid (e.g., oligonucleotide) labels, chemically reactive labels, fluorescent labels, enzyme labels, radioactive labels, and combinations thereof. Suitable detection antibodies for detecting the activation state and/or total amount of any of a variety of signal transduction molecules are available from Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.). As a non-limiting example, phospho-specific antibodies against various phosphorylated forms of signal transduction molecules such as EGFR, c-KIT, c-Src, FLK-1, PDGFRA, PDGFRB, AKT, MAPK, PTEN, Raf, and MEK are available from Santa Cruz Biotechnology.

The term “activation state-dependent antibody” includes a detection antibody which is specific for (i.e., binds, is bound by, or forms a complex with) a particular activation state of one or more analytes of interest in a sample. In preferred embodiments, the activation state-dependent antibody detects the phosphorylation, ubiquitination, and/or complexation state of one or more analytes such as one or more signal transduction molecules. In some embodiments, the phosphorylation of members of the EGFR family of receptor tyrosine kinases and/or the formation of heterodimeric complexes between EGFR family members is detected using activation state-dependent antibodies. In particular embodiments, activation state-dependent antibodies are useful for detecting one or more sites of phosphorylation in one or more of the following signal transduction molecules (phosphorylation sites correspond to the position of the amino acid in the human protein sequence): EGFR/HER1/ErbB1 (e.g., tyrosine (Y) 1068); ErbB2/HER2 (e.g., Y1248); ErbB3/HER3 (e.g., Y1289); ErbB4/HER4 (e.g., Y1284); c-Met (e.g., Y1003, Y1230, Y1234, Y1235, and/or Y1349); SGK3 (e.g., threonine (T) 256 and/or serine (S) 422); 4E-BP1 (e.g., T70); ERK1 (e.g., T185, Y187, T202, and/or Y204); ERK2 (e.g., T185, Y187, T202, and/or Y204); MEK (e.g., S217 and/or S221); PIK3R1 (e.g., Y688); PDK1 (e.g., S241); P70S6K (e.g., T229, T389, and/or S421); PTEN (e.g., S380); AKT1 (e.g., S473 and/or T308); AKT2 (e.g., S474 and/or T309); AKT3 (e.g., S472 and/or T305); GSK-33 (e.g., S9); NFKB (e.g., S536); IKB (e.g., S32); BAD (e.g., S112 and/or S136); mTOR (e.g., S2448); Rsk-1 (e.g., T357 and/or S363); Jnk (e.g., T183 and/or Y185); P38 (e.g., T180 and/or Y182); STAT3 (e.g., Y705 and/or S727); FAK (e.g., Y397, Y576, 5722, Y861, and/or S910); RB (e.g., S249, T252, 5612, and/or S780); RB1 (e.g., S780); adducin (e.g., S662 and/or S724); PYK2 (e.g., Y402 and/or Y881); PKCα(e.g., S657); PKCα/β (e.g., T368 and/or T641); PKCδ (e.g., T505); p53 (e.g., S392 and/or S20); CREB (e.g., S133); c-Jun (e.g., S63); c-Src (e.g., Y416); and paxillin (e.g., Y31 and/or Y118).

The term “activation state-independent antibody” includes a detection antibody which is specific for (i.e., binds, is bound by, or forms a complex with) one or more analytes of interest in a sample irrespective of their activation state. For example, the activation state-independent antibody can detect both phosphorylated and unphosphorylated forms of one or more analytes such as one or more signal transduction molecules.

The term “incubating” is used synonymously with “contacting” and “exposing” and does not imply any specific time or temperature requirements unless otherwise indicated.

The term “EGFR inhibitor-sensitive cell” includes a cell such as a colorectal cancer cell in which the expression and/or activation of EGFR is reduced or inhibited upon exposure to an EGFR inhibitor such as, e.g., cetuximab. Similarly, the term “EGFR inhibitor-sensitive subject” includes a subject having a cancer such as colorectal cancer in which the expression and/or activation of EGFR in the cancer cells is reduced or inhibited upon treatment with an EGFR inhibitor such as, e.g., cetuximab.

“Receptor tyrosine kinases” or “RTKs” include a family of fifty-six (56) proteins characterized by a transmembrane domain and a tyrosine kinase motif. RTKs function in cell signaling and transmit signals regulating growth, differentiation, adhesion, migration, and apoptosis. The mutational activation and/or overexpression of receptor tyrosine kinases transforms cells and often plays a crucial role in the development of cancers. RTKs have become targets of various molecularly targeted agents such as trastuzumab, cetuximab, gefitinib, erlotinib, sunitinib, imatinib, nilotinib, and the like. One well-characterized signal transduction pathway is the MAP kinase pathway, which is responsible for transducing the signal from epidermal growth factor (EGF) to the promotion of cell proliferation in cells.

### III. Description of the Embodiments

In certain aspects, the present invention provides methods for selecting a subject as suitable for combination therapy with both an EGFR (ErbB1) inhibitor and a HER2 inhibitor. In other aspects, the present invention provides methods for predicting whether a subject will benefit from combination therapy. In particular embodiments of the invention, methods are provided for determining whether to administer a combination therapy in a subject receiving therapy with an EGFR inhibitor. In further aspects, the present invention provides methods for monitoring a subject receiving therapy with an EGFR inhibitor to determine whether to administer a combination therapy comprising the EGFR inhibitor with a HER2 inhibitor.

In particular aspects, the present invention provides molecular markers (biomarkers) that enable the determination or prediction of whether a colorectal cancer can respond or is likely to respond favorably to a combination of anticancer drugs. In specific embodiments, measuring the level of expression and/or activation of at least one or more of HER1, HER2, HER3, PI3K, cMET, cKIT, IGF-1R, AKT, ERK, MEK, RSK, and/or SHC is particularly useful for selecting a suitable therapeutic regimen and/or monitoring therapy for a cancer such as colorectal cancer and/or identifying or predicting a response thereto in cancer cells (e.g., isolated cancer cells from a colorectal tumor). In some embodiments, measuring the formation of heterodimers and homodimers of HER1, HER2, and HER3 is particularly useful for selecting a suitable therapeutic regimen and/or monitoring therapy for a cancer such as colorectal cancer and/or identifying or predicting a response thereto in cancer cells (e.g., isolated cancer cells from a colorectal tumor). In some embodiments, measuring the binding of HER1, HER2, or HER3 to phosphoinositide 3-kinases (PI3K) is particularly useful for selecting a suitable therapeutic regimen and/or monitoring therapy for a cancer such as colorectal cancer and/or identifying or predicting a response thereto in cancer cells (e.g., isolated cancer cells from a colorectal tumor). In some embodiments, binding of HER3 to PI3K is measured.

In one aspect, the present invention provides a method for determining whether to administer combination therapy in a subject receiving therapy with an EGFR inhibitor, the method comprising:
(a) detecting and/or quantifying the level of a complex in a sample taken from the subject, wherein the complex comprises an ErbB dimer, a HER3:PI3K complex, or a combination thereof; and
(b) determining whether to administer a combination therapy comprising an EGFR inhibitor and a HER2 inhibitor based upon the level of the complex in the sample.

In some embodiments, the subject has colorectal cancer. In other embodiments, the subject is sensitive to an EGFR inhibitor such as, e.g., cetuximab. In yet other embodiments, the ErbB dimer is a receptor dimer including, e.g., HER2:HER2; HER3:HER3; HER2:HER3; HER1:HER2; HER1:HER3; HER2:HER4; HER3:HER4; p95HER2:HER3; p95HER2:HER2; truncated HER3 receptor with HER1, HER2, HER3, or HER4; and combinations thereof. In particular embodiments, the ErbB dimer is a receptor heterodimer such as, e.g., HER2:HER3.

In certain embodiments, step (a) comprises detecting and/or quantifying the level of the ErbB dimer and the level of the HER3:PI3K complex. In particular instances, the subject should be administered the combination therapy when the level of one or both complexes in the subject's sample is higher than a reference level thereof. In preferred embodiments, the subject should be administered the combination therapy when the levels of both complexes (i.e., both the ErbB dimer and the HER3:PI3K complex) in the subject's sample are higher than the reference levels thereof. In certain instances, the reference level is the level of the complex in a sample taken from the subject prior to EGFR inhibitor therapy or at an earlier time during EGFR inhibitor therapy. In certain other instances, the reference level is the level of the complex in a human cancer cell line (e.g., Lim1215 human colon cancer cells) without the EGFR inhibitor or at an early time point in the presence of the EGFR inhibitor.

In some embodiments, the method further comprises detecting and/or quantifying the expression and/or activation (e.g., phosphorylation) level of HER2 and/or HER3 in the sample. In particular instances, the subject should be administered the combination therapy when the expression and/or activation level of HER2 and/or HER3 in the sample is higher than a reference expression and/or activation level of HER2 and/or HER3. In one preferred embodiment, the level of HER2 expression, HER3 expression, and/or HER3 activation (e.g., phosphorylation) is higher than the reference level. In some instances, the reference level is the level of the expression and/or activation of HER2 and/or HER3 in a sample taken from the subject either prior to EGFR inhibitor therapy or at an earlier time during EGFR inhibitor therapy. In other instances, the reference level is the level of the expression and/or activation of HER2 and/or HER3 in a human cancer cell line (e.g., Lim1215 human colon cancer cells) without the EGFR inhibitor or at an early time point in the presence of the EGFR inhibitor.

In particular embodiments, the administration of the combination therapy reduces and/or inhibits the formation of the ErbB dimer and/or the HER3:PI3K complex. In other embodiments, the combination therapy also or alternatively reduces and/or inhibits HER2 expression, HER3 expression, and/or HER3 activation (e.g., phosphorylation).

Non-limiting examples of EGFR (ErbB1 or HER1) inhibitors include monoclonal antibodies such as cetuximab (Erbitux®), panitumumab (Vectibix™), matuzumab (EMD-72000), nimotuzumab, and zalutumumab; small molecule tyrosine kinase inhibitors such as gefitinib (Iressa®), erlotinib (Tarceva®), lapatinib (GW-572016; Tykerb®), canertinib (CI 1033), vandetanib (ZACTIMA™), pelitinib (EKB-569), CL-387785, neratinib (HKI-272), HKI-357, afatinib (BIBW-2992), varlitinib (ARRY-334543), and JNJ-26483327; ErbB1 vaccines; and combinations thereof. In particular embodiments, the EGFR inhibitor is cetuximab, gefitinib, erlotinib, or combinations thereof.

Non-limiting examples of HER2 (ErbB2) inhibitors include monoclonal antibodies such as trastuzumab (Herceptin®) and pertuzumab (2C4); small molecule tyrosine kinase inhibitors such as lapatinib (GW-572016; Tykerb®), gefitinib (Iressa®), erlotinib (Tarceva®), pelitinib (EKB-569), CP-654577, CP-724714, canertinib (CI 1033), HKI-272, PKI-166, AEE788, BMS-599626, HKI-357, afatinib (BIBW-2992), varlitinib (ARRY-334543), and JNJ-26483327; and combinations thereof. In particular embodiments, the HER2 inhibitor is trastuzumab, pertuzumab, or combinations thereof.

In particular embodiments, the combination therapy comprises a dual EGFR/HER2 inhibitor such as lapatinib (Tykerb®).

In some embodiments, the method further comprises determining or recommending that the subject be administered (in addition to the combination therapy or as an alternative to the combination therapy) a HER3 inhibitor and/or PI3K inhibitor.

Non-limiting examples of HER3 (ErbB3) inhibitors include monoclonal antibodies targeting the HER3 receptor such as pertuzumab (2C4), patritumab (U3-1287), GSK2849330, R05479599, AV-203, MM-121/SAR256212, MM-111, LJM716, and combinations thereof.

Non-limiting examples of PI3K inhibitors include BYL-719, BKM-120, PX-866, wortmannin, LY 294002, quercetin, tetrodotoxin citrate, thioperamide maleate, GDC-0941 (957054-30-7), IC87114, PI-103, PIK93, BEZ235 (NVP-BEZ235), TGX-115, ZSTK474, (−)-deguelin, NU 7026, myricetin, tandutinib, GDC-0941 bismesylate, GSK690693, KU-55933, MK-2206, OSU-03012, perifosine, triciribine, XL-147, PIK75, TGX-221, NU 7441, PI 828, XL-765, WHI-P 154, and combinations thereof.

In other embodiments, the method further comprises detecting and/or quantifying the expression (e.g., total amount) levels and/or activation (e.g., phosphorylation) levels in a tumor tissue sample of one or more additional signal transduction molecules such as HER1, p95HER2, cMET, cKIT, IGF-1R, VEGFR, PDGFR, PRAS, RPS6, SHC, AKT, ERK, PRAS, RPS6, MEK, RSK, 4EBP1, p70S6K, and combinations thereof. In certain embodiments, the method further comprises determining or recommending that the subject be administered (in addition to the combination therapy or as an alternative to the combination therapy) a pan-HER inhibitor, MEK inhibitor, and/or c-Met inhibitor based upon the levels of expression and/or activation of one or more of these molecules.

Non-limiting examples of pan-HER inhibitors include PF-00299804, neratinib (HKI-272), AC480 (BMS-599626), BMS-690154, PF-02341066, HM781-36B, CI-1033, BIBW-2992, and combinations thereof.

Non-limiting examples of MEK inhibitors include AS703026, PD98059, ARRY-162, RDEA119, U0126, GDC-0973, PD184161, AZD6244, AZD8330, PD0325901, ARRY-142886, and combinations thereof.

Non-limiting examples of c-Met inhibitors include monoclonal antibodies such as AMG102 and MetMAb; small molecule inhibitors of c-Met such as ARQ197, JNJ-38877605, PF-04217903, SGX523, GSK 1363089/XL880, XL184, MGCD265, and MK-2461; and combinations thereof.

In some embodiments, the sample is a cancer cell obtained from a subject's tumor, e.g., as a fine needle aspirate (FNA). In certain instances, the tumor is primary tumor tissue or metastatic tumor tissue.

In particular embodiments, the expression and/or activation levels of the dimers, complexes, and signal transduction molecules in the sample are measured, detected, and/or quantified by a Collaborative Enzyme Enhanced Reactive Immunoassay (CEER). The CEER technology is described in the following published patent documents, which are each herein incorporated by reference in their entirety for all purposes: PCT Patent Publication Nos. WO 2008/036802, WO 2009/012140, WO 2009/108637, WO 2010/132723, WO 2011/008990, WO 2011/050069, WO 2012/088337, and WO 2013/033623.

In another aspect, the present invention provides a method for monitoring a subject receiving therapy with an EGFR inhibitor, the method comprising:
(a) detecting and/or quantifying the level of a complex in a sample taken from the subject at time (t₂), wherein the complex comprises an ErbB dimer, a HER3:PI3K complex, or a combination thereof; and
(b) comparing the level of the complex detected and/or quantified at (t₂) to the level of the complex detected and/or quantified at an earlier time (t₁); and
(c) determining whether to administer a combination therapy comprising the EGFR inhibitor with a HER2 inhibitor based upon a difference between the level of the complex at (t₂) compared to (t₁).

In some embodiments, the subject has colorectal cancer. In other embodiments, the subject is sensitive to an EGFR inhibitor such as, e.g., cetuximab. In yet other embodiments, the ErbB dimer is a receptor dimer including, e.g., HER2:HER2; HER3:HER3; HER2:HER3; HER1:HER2; HER1:HER3; HER2:HER4; HER3:HER4; p95HER2:HER3; p95HER2:HER2; truncated HER3 receptor with HER1, HER2, HER3, or HER4; and combinations thereof. In particular embodiments, the ErbB dimer is a receptor heterodimer such as, e.g., HER2:HER3.

In certain embodiments, step (a) comprises detecting and/or quantifying the level of the ErbB dimer and the level of the HER3:PI3K complex. In particular instances, the subject should be administered the combination therapy when the level of one or both complexes in the subject's sample is higher at (t₂) compared to (t₁). In preferred embodiments, the subject should be administered the combination therapy when the levels of both complexes (i.e., both the ErbB dimer and HER3:PI3K complex) in the subject's sample are higher at (t₂) compared to (t₁). In certain embodiments, (t₁) corresponds to a time before, or shortly after, initiation of treatment with the EGFR inhibitor. In some instances, (t₁) corresponds to a time within about 0.5, 1, 2, 3, 4, 5, 6, 8, 12, 16, 20, or 24 hours prior to initiation of EGFR inhibitor therapy. In other instances, (t₁) corresponds to a time within about 0.5, 1, 2, 3, 4, 5, 6, 8, 12, 16, 20, or 24 hours after initiation of EGFR inhibitor therapy. In yet other instances, (t₂) corresponds to a time between about 24 hours to about 12 months after initiation of treatment with the EGFR inhibitor (e.g., about 1, 2, 3, 4, 5, 6, or 7 days, or about 1, 2, 3, 4, 5, 6, 7, or 8 weeks, or about 1, 2, 3, 4, 5, 6, 7, 8, 9, 10, 11, or 12 months post-treatment).

In some embodiments, the method further comprises detecting and/or quantifying the expression and/or activation (e.g., phosphorylation) level of HER2 and/or HER3 in the sample. In particular instances, the subject should be administered the combination therapy when the expression and/or activation level of HER2 and/or HER3 in the sample is higher at (t₂) compared to (t₁). In one preferred embodiment, the level of HER2 expression, HER3 expression, and/or HER3 activation (e.g., phosphorylation) is higher at (t₂) compared to (t₁).

In particular embodiments, the administration of the combination therapy reduces and/or inhibits the formation of the ErbB dimer and/or the HER3:PI3K complex. In other embodiments, the combination therapy also or alternatively reduces and/or inhibits HER2 expression, HER3 expression, and/or HER3 activation (e.g., phosphorylation).

Non-limiting examples of EGFR (ErbB1 or HER1) inhibitors include monoclonal antibodies such as cetuximab (Erbitux®), panitumumab (Vectibix™), matuzumab (EMD-72000), nimotuzumab, and zalutumumab; small molecule tyrosine kinase inhibitors such as gefitinib (Iressa®), erlotinib (Tarceva®), lapatinib (GW-572016; Tykerb®), canertinib (CI 1033), vandetanib (ZACTIMA™), pelitinib (EKB-569), CL-387785, neratinib (HKI-272), HKI-357, afatinib (BIBW-2992), varlitinib (ARRY-334543), and JNJ-26483327; ErbB1 vaccines; and combinations thereof. In particular embodiments, the EGFR inhibitor is cetuximab, gefitinib, erlotinib, or combinations thereof.

Non-limiting examples of HER2 (ErbB2) inhibitors include monoclonal antibodies such as trastuzumab (Herceptin®) and pertuzumab (2C4); small molecule tyrosine kinase inhibitors such as lapatinib (GW-572016; Tykerb®), gefitinib (Iressa®), erlotinib (Tarceva®), pelitinib (EKB-569), CP-654577, CP-724714, canertinib (CI 1033), HKI-272, PKI-166, AEE788, BMS-599626, HKI-357, afatinib (BIBW-2992), varlitinib (ARRY-334543), and JNJ-26483327; and combinations thereof. In particular embodiments, the HER2 inhibitor is trastuzumab, pertuzumab, or combinations thereof.

In particular embodiments, the combination therapy comprises a dual EGFR/HER2 inhibitor such as lapatinib (Tykerb®).

In some embodiments, the method further comprises determining or recommending that the subject be administered (in addition to the combination therapy or as an alternative to the combination therapy) a HER3 inhibitor and/or PI3K inhibitor.

Non-limiting examples of HER3 (ErbB3) inhibitors include monoclonal antibodies targeting the HER3 receptor such as pertuzumab (2C4), patritumab (U3-1287), GSK2849330, R05479599, AV-203, MM-121/SAR256212, MM-111, LJM716, and combinations thereof.

Non-limiting examples of PI3K inhibitors include BYL-719, BKM-120, PX-866, wortmannin, LY 294002, quercetin, tetrodotoxin citrate, thioperamide maleate, GDC-0941 (957054-30-7), IC87114, PI-103, PIK93, BEZ235 (NVP-BEZ235), TGX-115, ZSTK474, (−)-deguelin, NU 7026, myricetin, tandutinib, GDC-0941 bismesylate, GSK690693, KU-55933, MK-2206, OSU-03012, perifosine, triciribine, XL-147, PIK75, TGX-221, NU 7441, PI 828, XL-765, WHI-P 154, and combinations thereof.

In other embodiments, the method further comprises detecting and/or quantifying the expression (e.g., total amount) levels and/or activation (e.g., phosphorylation) levels in a tumor tissue sample of one or more additional signal transduction molecules such as HER1, p95HER2, cMET, cKIT, IGF-1R, VEGFR, PDGFR, PRAS, RPS6, SHC, AKT, ERK, PRAS, RPS6, MEK, RSK, 4EBP1, p70S6K, and combinations thereof. In certain embodiments, the method further comprises determining or recommending that the subject be administered (in addition to the combination therapy or as an alternative to the combination therapy) a pan-HER inhibitor, MEK inhibitor, and/or c-Met inhibitor based upon the levels of expression and/or activation of one or more of these molecules.

Non-limiting examples of pan-HER inhibitors include PF-00299804, neratinib (HKI-272), AC480 (BMS-599626), BMS-690154, PF-02341066, HM781-36B, CI-1033, BIBW-2992, and combinations thereof.

Non-limiting examples of MEK inhibitors include AS703026, PD98059, ARRY-162, RDEA119, U0126, GDC-0973, PD184161, AZD6244, AZD8330, PD0325901, ARRY-142886, and combinations thereof.

Non-limiting examples of c-Met inhibitors include monoclonal antibodies such as AMG102 and MetMAb; small molecule inhibitors of c-Met such as ARQ197, JNJ-38877605, PF-04217903, SGX523, GSK 1363089/XL880, XL184, MGCD265, and MK-2461; and combinations thereof.

In some embodiments, the sample is a cancer cell obtained from a subject's tumor, e.g., as a fine needle aspirate (FNA). In certain instances, the tumor is primary tumor tissue or metastatic tumor tissue.

In particular embodiments, the expression and/or activation levels of the dimers, complexes, and signal transduction molecules in the sample are measured, detected, and/or quantified by a Collaborative Enzyme Enhanced Reactive Immunoassay (CEER). The CEER technology is described in the following published patent documents, which are each herein incorporated by reference in their entirety for all purposes: PCT Patent Publication Nos. WO 2008/036802, WO 2009/012140, WO 2009/108637, WO 2010/132723, WO 2011/008990, WO 2011/050069, WO 2012/088337, and WO 2013/033623.

In some embodiments, the expression level and/or activation level of the analytes of interest (e.g., HER2, HER3, dimers thereof such as a HER2:HER3 dimer, complexes thereof such as a HER3:PI3K complex, etc.) is expressed as a relative fluorescence unit (RFU) value that corresponds to the signal intensity for a particular analyte of interest determined using, e.g., a proximity assay such as CEER. In other embodiments, the expression level and/or activation level of the one or more analytes is expressed as “−”, “±”, “+”, “++”, “+++”, or “++++” that corresponds to the increasing signal intensity for a particular analyte of interest that is determined using, e.g., a proximity assay such as CEER. In some instances, an undetectable or minimally detectable level of expression or activation of a particular analyte of interest that is determined using, e.g., a proximity assay such as CEER, may be expressed as “−” or “±”. In other instances, a low level of expression or activation of a particular analyte of interest that is determined using, e.g., a proximity assay such as CEER, may be expressed as “+”. In yet other instances, a moderate level of expression or activation of a particular analyte of interest that is determined using, e.g., a proximity assay such as CEER, may be expressed as “++”. In still yet other instances, a high level of expression or activation of a particular analyte of interest that is determined using, e.g., a proximity assay such as CEER, may be expressed as “+++”. In further instances, a very high level of expression or activation of a particular analyte of interest that is determined using, e.g., a proximity assay such as CEER, may be expressed as “++++”.

In other embodiments, the expression level and/or activation level of the analytes of interest (e.g., HER2, HER3, dimers thereof such as a HER2:HER3 dimer, complexes thereof such as a HER3:PI3K complex, etc.) is quantitated by calibrating or normalizing the RFU value that is determined using, e.g., a proximity assay such as CEER, against a standard curve generated for the particular analyte of interest. In certain instances, a computed units (CU) value can be calculated based upon the standard curve. In other instances, the CU value can be expressed as “−”, “±”, “+”, “++”, “+++”, or “++++” in accordance with the description above for signal intensity.

In certain embodiments, the expression or activation level of a particular analyte of interest (e.g., HER2, HER3, dimers thereof such as a HER2:HER3 dimer, complexes thereof such as a HER3:PI3K complex, etc.) corresponds to a level of expression or activation that is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or 100-fold higher or lower (e.g., about 1.5-3, 2-3, 2-4, 2-5, 2-10, 2-20, 2-50, 3-5, 3-10, 3-20, 3-50, 4-5, 4-10, 4-20, 4-50, 5-10, 5-15, 5-20, or 5-50-fold higher or lower) than a reference expression or activation level of the analyte of interest, e.g., when compared to an expression or activation level of the analyte of interest determined in tumor tissue from a subject sensitive to an EGFR inhibitor prior to EGFR inhibitor treatment, at an earlier time in EGFR inhibitor therapy, after receiving combination therapy with an EGFR inhibitor and HER2 inhibitor, or prior to the combination therapy, or when compared to an expression or activation level of the analyte of interest determined in a cancer cell line (e.g., human colon cancer cell line such as Lim1215 cells) in the absence of an EGFR inhibitor, at an early time point in the presence of the EGFR inhibitor, in the presence of both the EGFR inhibitor and the HER2 inhibitor, or in the presence of the EGFR inhibitor only.

In particular embodiments, the expression level or activation level of the analyte of interest (e.g., HER2, HER3, dimers thereof such as a HER2:HER3 dimer, complexes thereof such as a HER3:PI3K complex, etc.) is higher in tumor tissue from a subject receiving EGFR inhibitor treatment (e.g., monotherapy with cetuximab) when compared to tumor tissue from the subject prior to EGFR inhibitor treatment or at an earlier time in EGFR inhibitor therapy, or when compared to a cancer cell line (e.g., a human colon cancer cell line such as Lim1215 cells) in the absence of the EGFR inhibitor or at an early time point (e.g., 0, 0.5, 1, 2, 4, 6, 8, 12 hours) in the presence of the EGFR inhibitor (e.g., incubating cells from a cancer cell line in vitro with cetuximab).

In particular embodiments, the expression level or activation level of the analyte of interest (e.g., HER2, HER3, dimers thereof such as a HER2:HER3 dimer, complexes thereof such as a HER3:PI3K complex, etc.) is higher in tumor tissue from a subject receiving EGFR inhibitor treatment (e.g., monotherapy with cetuximab) when compared to tumor tissue from the subject after receiving combination therapy with an EGFR inhibitor and HER2 inhibitor (e.g., therapy with cetuximab and trastuzumab or with a dual EGFR/HER2 inhibitor such as lapatinib), or when compared to a cancer cell line (e.g., a human colon cancer cell line such as Lim1215 cells) in the presence of both the EGFR inhibitor and the HER2 inhibitor (e.g., incubating cells from a cancer cell line in vitro with both cetuximab and trastuzumab or with a dual EGFR/HER2 inhibitor such as lapatinib).

In particular embodiments, the expression level or activation level of the analyte of interest (e.g., HER2, HER3, dimers thereof such as a HER2:HER3 dimer, complexes thereof such as a HER3:PI3K complex, etc.) is lower in tumor tissue from a subject receiving EGFR inhibitor treatment together with HER2 inhibitor treatment (e.g., therapy with cetuximab and trastuzumab or with a dual EGFR/HER2 inhibitor such as lapatinib) when compared to tumor tissue from the subject prior to the combination therapy (e.g., monotherapy with cetuximab), or when compared to a cancer cell line (e.g., a human colon cancer cell line such as Lim1215 cells) in the absence of both the EGFR inhibitor and the HER2 inhibitor (e.g., incubating cells from a cancer cell line in vitro with cetuximab only).

In some aspects, the methods of the invention further comprise genotyping nucleic acid obtained from the sample to determine the presence or absence of a variant allele in an oncogene such as KRAS, BRAF, PIK3CA, and/or EGFR.

In particular embodiments, the methods of the present invention further comprise a step of genotyping for the presence or absence of a variant allele (e.g., somatic mutation) at a polymorphic site in an oncogene such as KRAS, BRAF, PIK3CA, and/or EGFR (e.g., one or more somatic mutations at one, two, three, four, five, six or more polymorphic sites such as a single nucleotide polymorphism (SNP)) in the sample.

The presence or absence of a variant allele (e.g., somatic mutation) in an oncogene of interest can be determined using any genotyping assay known in the art. Assays that can be used to determine somatic mutation or variant allele status include, but are not limited to, electrophoretic analysis, restriction length polymorphism analysis, sequence analysis, hybridization analysis, PCR analysis, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, denaturing gradient gel electrophoresis, and combinations thereof. These assays have been well-described and standard methods are known in the art. See, e.g., Ausubel et al., Current Protocols in Molecular Biology, John Wiley & Sons, Inc. New York (1984-2008), Chapter 7 and Supplement 47; Theophilus et al., “PCR Mutation Detection Protocols,” Humana Press, (2002); Innis et al., PCR Protocols, San Diego, Academic Press, Inc. (1990); Maniatis, et al., Molecular Cloning: A Laboratory Manual, Cold Spring Harbor Lab., New York, (1982); Ausubel et al., Current Protocols in Genetics and Genomics, John Wiley & Sons, Inc. New York (1984-2008); and Ausubel et al., Current Protocols in Human Genetics, John Wiley & Sons, Inc. New York (1984-2008); all incorporated herein by reference in their entirety for all purposes.

In certain instances, the methods of the invention may further comprise a step of providing the result of the combination therapy determination or recommendation to a user (e.g., a clinician such as an oncologist or a general practitioner) in a readable format. In some instances, the method may further comprise sending or reporting the result of the combination therapy determination or recommendation to a clinician, e.g., an oncologist or a general practitioner. In other instances, the method may further comprise recording or storing the result of the combination therapy determination or recommendation in a computer database or other suitable machine or device for storing information, e.g., at a laboratory.

To preserve the in situ activation states, signal transduction proteins are typically extracted shortly after the cells are isolated, preferably within 96, 72, 48, 24, 6, or 1 hr, more preferably within 30, 15, or 5 minutes. The isolated cells may also be incubated with growth factors usually at nanomolar to micromolar concentrations for about 1-30 minutes to resuscitate or stimulate signal transducer activation (see, e.g., Irish et al., Cell, 118:217-228 (2004)). Stimulatory growth factors include epidermal growth factor (EGF), heregulin (HRG), TGF-α, PIGF, angiopoietin (Ang), NRG1, PGF, TNF-α, VEGF, PDGF, IGF, FGF, HGF, cytokines, and the like. To evaluate potential anticancer therapies for an individual patient, the isolated cells can be incubated with one or more anticancer drugs of varying doses prior to, during, and/or after growth factor stimulation. Growth factor stimulation can be performed for a few minutes or hours (e.g., about 1-5 minutes to about 1-6 hours). After isolation, treatment with the anticancer drug, and/or growth factor stimulation, the cells are lysed to extract the signal transduction proteins using any technique known in the art. Preferably, the cell lysis is initiated between about 1-360 minutes after growth factor stimulation, and more preferably at two different time intervals: (1) at about 1-5 minutes after growth factor stimulation; and (2) between about 30-180 minutes after growth factor stimulation. Alternatively, the lysate can be stored at −80° C. until use.

In some embodiments, determining the expression level of the one or more analytes comprises detecting the total amount of each of the one or more analytes in the cellular extract with one or more antibodies specific for the corresponding analyte. In particular embodiments, the antibodies bind to the analyte irrespective of the activation state of the analyte to be detected, i.e., the antibodies detect both the non-activated and activated forms of the analyte.

Total expression level and/or status can be determined using any of a variety of techniques. In certain embodiments, the total expression level and/or status of each of the one or more analytes such as signal transduction molecules in a sample is detected with an immunoassay (e.g., ELISA or CEER), a homogeneous mobility shift assay (HMSA), or an immunohistochemical assay.

Non-limiting examples of ELISA kits for detecting the presence or level of analytes of interest in a sample are available from, e.g., Antigenix America Inc. (Huntington Station, N.Y.), Promega (Madison, Wis.), R&D Systems, Inc. (Minneapolis, Minn.), Invitrogen (Camarillo, Calif.), Neogen Corp. (Lexington, Ky.), CHEMICON International, Inc. (Temecula, Calif.), PeproTech (Rocky Hill, N.J.), Alpco Diagnostics (Salem, N.H.), Pierce Biotechnology, Inc. (Rockford, Ill.), and/or Abazyme (Needham, Mass.).

In particular embodiments, the presence or level of analytes of interest is detected using a multiplexed immunoarray, such as CEER, also known as the Collaborative Proximity Immunoassay (COPIA). CEER is described in the following patent documents which are each herein incorporated by reference in their entirety for all purposes: International Patent Publication Nos. WO 2008/036802, WO 2009/012140, WO 2009/108637, WO 2010/132723, WO 2011/008990, WO 2011/050069; WO 2012/088337; WO 2012/119113; and WO 2013/033623.

In particular embodiments, the presence or level of analytes of interest is detected with a homogeneous mobility shift assay (HMSA) using size exclusion chromatography. These methods and related technology are described in International Patent Publication Nos. WO 2011/056590, WO 2012/054532, WO 2012/154253 and WO 2013/006810, and in U.S. Provisional Application No. 61/683,681, filed Aug. 15, 2012, the disclosures of which are incorporated by reference in their entirety for all purposes.

In certain embodiments, determining the expression (e.g., total) levels of the one or more analytes comprises:
(i) incubating (e.g., contacting) a cellular extract produced from the cell with one or a plurality of dilution series of capture antibodies (e.g., capture antibodies specific for one or more analytes) to form a plurality of captured analytes, wherein the capture antibodies are restrained on a solid support (e.g., to transform the analytes present in the cellular extract into complexes of captured analytes comprising the analytes and capture antibodies);
(ii) incubating (e.g., contacting) the plurality of captured analytes with detection antibodies comprising one or a plurality of first and second activation state-independent antibodies specific for the corresponding analytes (e.g., first and second activation state-independent antibodies specific for the one or more analytes) to form a plurality of detectable captured analytes (e.g., to transform the complexes of captured analytes into complexes of detectable captured analytes comprising the captured analytes and detection antibodies),
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(iii) incubating (e.g., contacting) the plurality of detectable captured analytes with a second member of the signal amplification pair to generate an amplified signal; and
(iv) detecting the amplified signal generated from the first and second members of the signal amplification pair.

In certain other embodiments, determining the expression (e.g., total) levels of the one or more analytes that are truncated receptors (e.g., p95HER2) comprises:
(i) incubating (e.g., contacting) a cellular extract produced from the cell with a plurality of beads specific for an extracellular domain (ECD) binding region of a full-length receptor (e.g., full-length HER2);
(ii) removing the plurality of beads from the cellular extract, thereby removing the full-length receptor (e.g., full-length HER2) to form a cellular extract devoid of the full-length receptor (e.g., full-length HER2) (e.g., to transform the cellular extract into a cellular extract devoid of a specific full-length receptor or family of full-length receptors);
(iii) incubating (e.g., contacting) the cellular extract devoid of the full-length receptor (e.g., full-length HER2) with one or a plurality of capture antibodies specific for an intracellular domain (ICD) binding region of the full-length receptor (e.g., full-length HER2) to form a plurality of captured truncated receptors, wherein the capture antibodies are restrained on a solid support (e.g., to transform the truncated receptors present in a full-length receptor-depleted cellular extract into complexes of truncated receptors and capture antibodies);
(iv) incubating the plurality of captured truncated receptors with detection antibodies comprising one or a plurality of first and second activation state-independent antibodies specific for an ICD binding region of the full-length receptor (e.g., full-length HER2) to form a plurality of detectable captured truncated receptors (e.g., to transform the complexes of captured truncated receptors into complexes of detectable captured truncated receptors comprising the captured truncated receptors and detection antibodies),
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(v) incubating (e.g., contacting) the plurality of detectable captured truncated receptors with a second member of the signal amplification pair to generate an amplified signal; and
(vi) detecting the amplified signal generated from the first and second members of the signal amplification pair.

The first activation state-independent antibodies may be directly labeled with the facilitating moiety or indirectly labeled with the facilitating moiety, e.g., via hybridization between an oligonucleotide conjugated to the first activation state-independent antibodies and a complementary oligonucleotide conjugated to the facilitating moiety. Similarly, the second activation state-independent antibodies may be directly labeled with the first member of the signal amplification pair or indirectly labeled with the first member of the signal amplification pair, e.g., via binding between a first member of a binding pair conjugated to the second activation state-independent antibodies and a second member of the binding pair conjugated to the first member of the signal amplification pair. In certain instances, the first member of the binding pair is biotin and the second member of the binding pair is an avidin such as streptavidin or neutravidin.

In some embodiments, the facilitating moiety may be, for example, glucose oxidase. In certain instances, the glucose oxidase and the first activation state-independent antibodies can be conjugated to a sulfhydryl-activated dextran molecule as described in, e.g., Examples 16-17 of PCT Publication No. WO2009/108637, the disclosure of which is herein incorporated by reference in its entirety for all purposes. The sulfhydryl-activated dextran molecule typically has a molecular weight of about 500 kDa (e.g., about 250, 300, 350, 400, 450, 500, 550, 600, 650, 700, or 750 kDa). In other embodiments, the oxidizing agent may be, for example, hydrogen peroxide (H₂O₂). In yet other embodiments, the first member of the signal amplification pair may be, for example, a peroxidase such as horseradish peroxidase (HRP). In further embodiments, the second member of the signal amplification pair may be, for example, a tyramide reagent (e.g., biotin-tyramide). Preferably, the amplified signal is generated by peroxidase oxidization of biotin-tyramide to produce an activated tyramide (e.g., to transform the biotin-tyramide into an activated tyramide). The activated tyramide may be directly detected or indirectly detected, e.g., upon the addition of a signal-detecting reagent. Non-limiting examples of signal-detecting reagents include streptavidin-labeled fluorophores and combinations of streptavidin-labeled peroxidases and chromogenic reagents such as, e.g., 3,3′,5,5′-tetramethylbenzidine (TMB).

In certain instances, the horseradish peroxidase and the second activation state-independent antibodies can be conjugated to a sulfhydryl-activated dextran molecule. The sulfhydryl-activated dextran molecule typically has a molecular weight of about 70 kDa (e.g., about 40, 45, 50, 55, 60, 65, 70, 75, 80, 85, 90, 95, or 100 kDa).

The truncated receptor is typically a fragment of the full-length receptor and shares an intracellular domain (ICD) binding region with the full-length receptor. In certain embodiments, the full-length receptor comprises an extracellular domain (ECD) binding region, a transmembrane domain, and an intracellular domain (ICD) binding region. Without being bound to any particular theory, the truncated receptor may arise through the proteolytic processing of the ECD of the full-length receptor or by alternative initiation of translation from methionine residues that are located before, within, or after the transmembrane domain, e.g., to create a truncated receptor with a shortened ECD or a truncated receptor comprising a membrane-associated or cytosolic ICD fragment.

In certain preferred embodiments, the truncated receptor is p95HER2 and the corresponding full-length receptor is HER2. However, one skilled in the art will appreciate that the methods described herein for detecting truncated proteins can be applied to a number of different proteins including, but not limited to, the EGFR VIII mutant (implicated in glioblastoma, colorectal cancer, etc.), other truncated receptor tyrosine kinases, caspases, and the like. Example 12 of PCT Publication No. WO2009/108637, the disclosure of which is herein incorporated by reference in its entirety for all purposes, provides an exemplary embodiment of the assay methods of the present invention for detecting truncated receptors such as p95HER2 in cells using a multiplex, high-throughput, proximity dual detection microarray ELISA having superior dynamic range.

In some embodiments, the plurality of beads specific for an ECD binding region comprises a streptavidin-biotin pair, wherein the streptavidin is attached to the bead and the biotin is attached to an antibody. In certain instances, the antibody is specific for the ECD binding region of the full-length receptor (e.g., full-length HER2).

In some embodiments, each dilution series of capture antibodies comprises a series of descending capture antibody concentrations. In certain instances, the capture antibodies are serially diluted at least 2-fold (e.g., 2, 5, 10, 20, 50, 100, 500, or 1000-fold) to produce a dilution series comprising a set number (e.g., 2, 3, 4, 5, 6, 7, 8, 9, 10, 15, 20, 25, or more) of descending capture antibody concentrations which are spotted onto an array. Preferably, at least 2, 3, 4, 5, or 6 replicates of each capture antibody dilution are spotted onto the array.

In other embodiments, the solid support comprises glass (e.g., a glass slide), plastic, chips, pins, filters, beads, paper, membrane (e.g., nylon, nitrocellulose, polyvinylidene fluoride (PVDF), etc.), fiber bundles, or any other suitable substrate. In a preferred embodiment, the capture antibodies are restrained (e.g., via covalent or noncovalent interactions) on glass slides coated with a nitrocellulose polymer such as, for example, FAST® Slides, which are commercially available from Whatman Inc. (Florham Park, N.J.). Exemplary methods for constructing antibody arrays suitable for use in the invention are described, e.g., in PCT Publication No. WO2009/108637, the disclosure of which is herein incorporated by reference in its entirety for all purposes.

In further embodiments, determining the activation levels of the one or more analytes comprises detecting a phosphorylation level of the one or more analytes in the cellular extract with antibodies specific for the phosphorylated form of each of the analytes to be detected.

Phosphorylation levels and/or status can be determined using any of a variety of techniques. For example, it is well known in the art that phosphorylated proteins can be detected via immunoassays using antibodies that specifically recognize the phosphorylated form of the protein (see, e.g., Lin et al., Br. J. Cancer, 93:1372-1381 (2005)) Immunoassays generally include immunoblotting (e.g., Western blotting), RIA, and ELISA. More specific types of immunoassays include antigen capture/antigen competition, antibody capture/antigen competition, two-antibody sandwiches, antibody capture/antibody excess, and antibody capture/antigen excess. Methods of making antibodies are described herein and in Harlow and Lane, Antibodies: A Laboratory Manual, 1988, Cold Spring Harbor Laboratory Press, Cold Spring Harbor, N.Y., USA. Phospho-specific antibodies can be made de novo or obtained from commercial or noncommercial sources. Phosphorylation levels and/or status can also be determined by metabolically labeling cells with radioactive phosphate in the form of [γ-³²P]ATP or [γ-³³P]ATP. Phosphorylated proteins become radioactive and hence traceable and quantifiable through scintillation counting, radiography, and the like (see, e.g., Wang et al., J. Biol. Chem., 253:7605-7608 (1978)). For example, metabolically labeled proteins can be extracted from cells, separated by gel electrophoresis, transferred to a membrane, probed with an antibody specific for a particular analyte and subjected to autoradiography to detect ³²P or ³³P. Alternatively, the gel can be subjected to autoradiography prior to membrane transference and antibody probing.

In particular embodiments, the activation (e.g., phosphorylation) level and/or status of each of the one or more analytes in a sample is detected with an immunoassay such as a proximity dual detection assay (e.g., CEER).

In certain embodiments, determining the activation (e.g., phosphorylation) level of the one or more analytes comprises:
(i) incubating (e.g., contacting) a cellular extract produced from a sample with a dilution series of capture antibodies (e.g., capture antibodies specific for one or more analytes) to form a plurality of captured analytes, wherein the capture antibodies are restrained on a solid support (e.g., to transform the analytes present in the cellular extract into complexes of captured analytes comprising the analytes and capture antibodies);
(ii) incubating (e.g., contacting) the plurality of captured analytes with detection antibodies comprising activation state-independent antibodies specific for the corresponding analytes (e.g., activation state-independent antibodies specific for the one or more analytes) and activation state-dependent antibodies specific for the corresponding analytes (e.g., activation state-dependent antibodies specific for the one or more analytes) to form a plurality of detectable captured analytes (e.g., to transform the complexes of captured analytes into complexes of detectable captured analytes comprising the captured analytes and detection antibodies),
wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair;
(iii) incubating (e.g., contacting) the plurality of detectable captured analytes with a second member of the signal amplification pair to generate an amplified signal; and
(iv) detecting the amplified signal generated from the first and second members of the signal amplification pair.

The activation state-independent antibodies may be directly labeled with the facilitating moiety or indirectly labeled with the facilitating moiety, e.g., via hybridization between an oligonucleotide conjugated to the activation state-independent antibodies and a complementary oligonucleotide conjugated to the facilitating moiety. Similarly, the activation state-dependent antibodies may be directly labeled with the first member of the signal amplification pair or indirectly labeled with the first member of the signal amplification pair, e.g., via binding between a first member of a binding pair conjugated to the activation state-dependent antibodies and a second member of the binding pair conjugated to the first member of the signal amplification pair. In certain instances, the first member of the binding pair is biotin and the second member of the binding pair is an avidin such as streptavidin or neutravidin.

In some embodiments, the facilitating moiety may be, for example, glucose oxidase. In certain instances, the glucose oxidase and the activation state-independent antibodies can be conjugated to a sulfhydryl-activated dextran molecule as described in, e.g., Examples 16-17 of PCT Publication No. WO2009/108637, the disclosure of which is herein incorporated by reference in its entirety for all purposes. The sulfhydryl-activated dextran molecule typically has a molecular weight of about 500 kDa (e.g., about 250, 300, 350, 400, 450, 500, 550, 600, 650, 700, or 750 kDa). In other embodiments, the oxidizing agent may be, for example, hydrogen peroxide (H₂O₂). In yet other embodiments, the first member of the signal amplification pair may be, for example, a peroxidase such as horseradish peroxidase (HRP). In further embodiments, the second member of the signal amplification pair may be, for example, a tyramide reagent (e.g., biotin-tyramide). Preferably, the amplified signal is generated by peroxidase oxidization of biotin-tyramide to produce an activated tyramide (e.g., to transform the biotin-tyramide into an activated tyramide). The activated tyramide may be directly detected or indirectly detected, e.g., upon the addition of a signal-detecting reagent. Non-limiting examples of signal-detecting reagents include streptavidin-labeled fluorophores and combinations of streptavidin-labeled peroxidases and chromogenic reagents such as, e.g., 3,3′,5,5′-tetramethylbenzidine (TMB).

In certain instances, the horseradish peroxidase and the activation state-dependent antibodies can be conjugated to a sulfhydryl-activated dextran molecule. The sulfhydryl-activated dextran molecule typically has a molecular weight of about 70 kDa (e.g., about 40, 45, 50, 55, 60, 65, 70, 75, 80, 85, 90, 95, or 100 kDa).

In some embodiments, each dilution series of capture antibodies comprises a series of descending capture antibody concentrations. In certain instances, the capture antibodies are serially diluted at least 2-fold (e.g., 2, 5, 10, 20, 50, 100, 500, or 1000-fold) to produce a dilution series comprising a set number (e.g., 2, 3, 4, 5, 6, 7, 8, 9, 10, 15, 20, 25, or more) of descending capture antibody concentrations which are spotted onto an array. Preferably, at least 2, 3, 4, 5, or 6 replicates of each capture antibody dilution are spotted onto the array.

In other embodiments, the solid support comprises glass (e.g., a glass slide), plastic, chips, pins, filters, beads, paper, membrane (e.g., nylon, nitrocellulose, polyvinylidene fluoride (PVDF), etc.), fiber bundles, or any other suitable substrate. In a preferred embodiment, the capture antibodies are restrained (e.g., via covalent or noncovalent interactions) on glass slides coated with a nitrocellulose polymer such as, for example, FAST® Slides, which are commercially available from Whatman Inc. (Florham Park, N.J.). Exemplary methods for constructing antibody arrays suitable for use in the invention are described, e.g., in PCT Publication No. WO2009/108637, the disclosure of which is herein incorporated by reference in its entirety for all purposes.

### IV. Single Detection Assays

In some embodiments, the assay for detecting the expression and/or activation level of one or more analytes of interest in a cellular extract of cells such as tumor cells is a multiplex, high-throughput two-antibody assay having superior dynamic range. As a non-limiting example, the two antibodies used in the assay can comprise: (1) a capture antibody specific for a particular analyte of interest; and (2) a detection antibody specific for an activated form of the analyte (i.e., activation state-dependent antibody). The activation state-dependent antibody is capable of detecting, for example, the phosphorylation, ubiquitination, and/or complexation state of the analyte. Alternatively, the detection antibody comprises an activation state-independent antibody, which detects the total amount of the analyte in the cellular extract. The activation state-independent antibody is generally capable of detecting both the activated and non-activated forms of the analyte.

In one particular embodiment, the two-antibody assay for detecting the expression or activation level of an analyte of interest comprises:
(i) incubating the cellular extract with one or a plurality of dilution series of capture antibodies to form a plurality of captured analytes;
(ii) incubating the plurality of captured analytes with detection antibodies specific for the corresponding analytes to form a plurality of detectable captured analytes, wherein the detection antibodies comprise activation state-dependent antibodies for detecting the activation (e.g., phosphorylation) level of the analyte or activation state-independent antibodies for detecting the expression level (e.g., total amount) of the analyte;
(iii) incubating the plurality of detectable captured analytes with first and second members of a signal amplification pair to generate an amplified signal; and
(iv) detecting the amplified signal generated from the first and second members of the signal amplification pair.

The two-antibody assays described herein are typically antibody-based arrays which comprise a plurality of different capture antibodies at a range of capture antibody concentrations that are coupled to the surface of a solid support in different addressable locations. Examples of suitable solid supports for use in the present invention are described above.

The capture antibodies and detection antibodies are preferably selected to minimize competition between them with respect to analyte binding (i.e., both capture and detection antibodies can simultaneously bind their corresponding signal transduction molecules).

In one embodiment, the detection antibodies comprise a first member of a binding pair (e.g., biotin) and the first member of the signal amplification pair comprises a second member of the binding pair (e.g., streptavidin). The binding pair members can be coupled directly or indirectly to the detection antibodies or to the first member of the signal amplification pair using methods well-known in the art. In certain instances, the first member of the signal amplification pair is a peroxidase (e.g., horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, etc.), and the second member of the signal amplification pair is a tyramide reagent (e.g., biotin-tyramide). In these instances, the amplified signal is generated by peroxidase oxidization of the tyramide reagent to produce an activated tyramide in the presence of hydrogen peroxide (H₂O₂).

The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor® dye (e.g., Alexa Fluor® 555), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS), 4-chloro-1-napthol (4CN), and/or porphyrinogen.

An exemplary protocol for performing the two-antibody assays described herein is provided in Example 3 of PCT Publication No. WO2009/108637, the disclosure of which is herein incorporated by reference in its entirety for all purposes.

In another embodiment of a two-antibody approach, the present invention provides a method for detecting the expression or activation level of a truncated receptor, the method comprising:
(i) incubating the cellular extract with a plurality of beads specific for an extracellular domain (ECD) binding region of a full-length receptor;
(ii) removing the plurality of beads from the cellular extract, thereby removing the full-length receptor to form a cellular extract devoid of the full-length receptor;
(iii) incubating the cellular extract devoid of the full-length receptor with a dilution series of one or a plurality of capture antibodies specific for an intracellular domain (ICD) binding region of the full-length receptor to form a plurality of captured truncated receptors;
(iv) incubating the plurality of captured truncated receptors with detection antibodies specific for an ICD binding region of the full-length receptor to form a plurality of detectable captured truncated receptors, wherein the detection antibodies comprise activation state-dependent antibodies for detecting the activation (e.g., phosphorylation) level of the truncated receptor or activation state-independent antibodies for detecting the expression level (e.g., total amount) of the truncated receptor;
(v) incubating the plurality of detectable captured truncated receptors with first and second members of a signal amplification pair to generate an amplified signal; and
(vi) detecting an amplified signal generated from the first and second members of the signal amplification pair.

In certain embodiments, the truncated receptor is p95HER2 and the full-length receptor is HER2. In certain other embodiments, the plurality of beads specific for an extracellular domain (ECD) binding region comprises a streptavidin-biotin pair, wherein the biotin is attached to the bead and the biotin is attached to an antibody (e.g., wherein the antibody is specific for the ECD binding region of the full-length receptor).

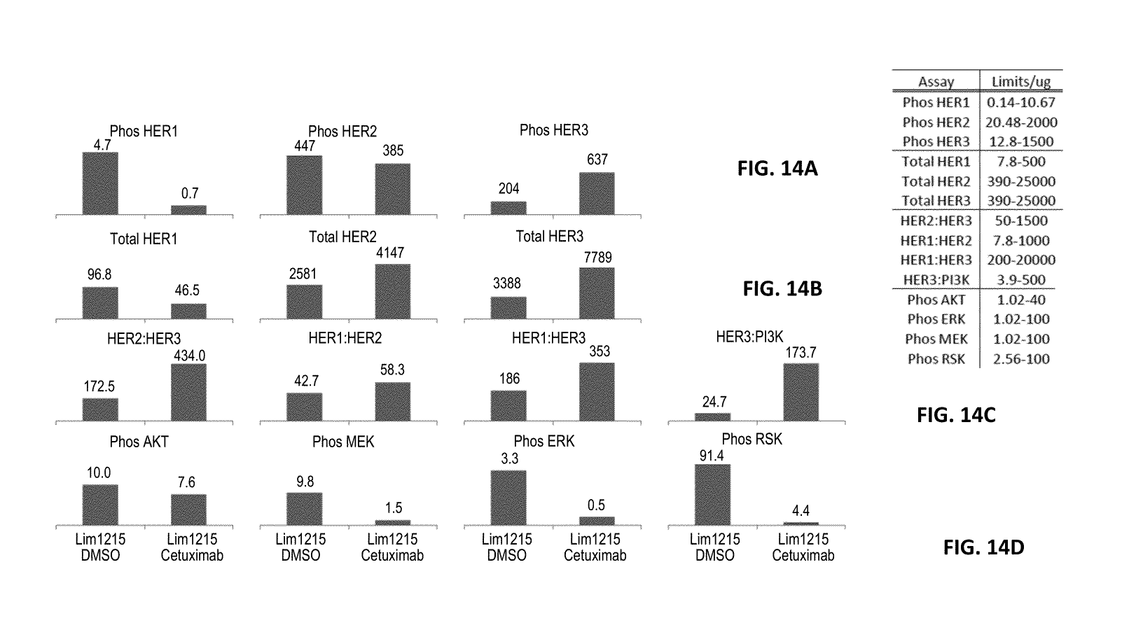

FIG. 14A of PCT Publication No. WO2009/108637, the disclosure of which is herein incorporated by reference in its entirety for all purposes, shows that beads coated with an antibody directed to the extracellular domain (ECD) of a receptor of interest binds the full-length receptor (e.g., HER2), but not the truncated receptor (e.g., p95HER2) to remove any full-length receptor from the assay. FIG. 14B of PCT Publication No. WO2009/108637 shows that the truncated receptor (e.g., p95HER2), once bound to a capture antibody, may then be detected by a detection antibody that is specific for the intracellular domain (ICD) of the full-length receptor (e.g., HER2). The detection antibody may be directly conjugated to horseradish peroxidase (HRP). Tyramide signal amplification (TSA) may then be performed to generate a signal to be detected. The expression level or activation state of the truncated receptor (e.g., p95HER2) can be interrogated to determine, e.g., its total concentration or its phosphorylation state, ubiquitination state, and/or complexation state.

In another embodiment, the present invention provides kits for performing the two-antibody assays described above comprising: (a) a dilution series of one or a plurality of capture antibodies restrained on a solid support; and (b) one or a plurality of detection antibodies (e.g., activation state-independent antibodies and/or activation state-dependent antibodies). In some instances, the kits can further contain instructions for methods of using the kit to detect the expression levels and/or activation states of one or a plurality of signal transduction molecules of cells such as tumor cells. The kits may also contain any of the additional reagents described above with respect to performing the specific methods of the present invention such as, for example, first and second members of the signal amplification pair, tyramide signal amplification reagents, wash buffers, etc.

### V. Proximity Dual Detection Assays

In some embodiments, the assay for detecting the expression and/or activation level of one or more analytes of interest in a cellular extract of cells such as tumor cells is a multiplex, high-throughput proximity (i.e., three-antibody) assay having superior dynamic range. As a non-limiting example, the three antibodies used in the proximity assay can comprise: (1) a capture antibody specific for a particular analyte of interest; (2) a detection antibody specific for an activated form of the analyte (i.e., activation state-dependent antibody); and (3) a detection antibody which detects the total amount of the analyte (i.e., activation state-independent antibody). The activation state-dependent antibody is capable of detecting, e.g., the phosphorylation, ubiquitination, and/or complexation state of the analyte, while the activation state-independent antibody is capable of detecting the total amount (i.e., both the activated and non-activated forms) of the analyte. As another example, the three antibodies used in the proximity assay can comprise: (1) a capture antibody specific for a particular analyte complex of interest (such as, e.g., a HER1:HER2 dimer); (2) a detection antibody specific for a first component of the complex; and (3) a detection antibody which detects a second component of the complex. Detection assays for ErbB dimerization and PI3K complexes are described, for example, in PCT Publication No. WO 2013/033623.

In one particular embodiment, the proximity assay for detecting the activation level or status of an analyte of interest comprises:
(i) incubating the cellular extract with one or a plurality of dilution series of capture antibodies to form a plurality of captured analytes;
(ii) incubating the plurality of captured analytes with detection antibodies comprising one or a plurality of activation state-independent antibodies and one or a plurality of activation state-dependent antibodies specific for the corresponding analytes to form a plurality of detectable captured analytes,
wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair;
(iii) incubating the plurality of detectable captured analytes with a second member of the signal amplification pair to generate an amplified signal; and
(iv) detecting the amplified signal generated from the first and second members of the signal amplification pair.

In another particular embodiment, the proximity assay for detecting the activation level or status of an analyte of interest that is a truncated receptor comprises:
(i) incubating the cellular extract with a plurality of beads specific for an extracellular domain (ECD) binding region of a full-length receptor;
(ii) removing the plurality of beads from the cellular extract, thereby removing the full-length receptor to form a cellular extract devoid of the full-length receptor;
(iii) incubating the cellular extract devoid of the full-length receptor with one or a plurality of capture antibodies specific for an intracellular domain (ICD) binding region of the full-length receptor to form a plurality of captured truncated receptors;
(iv) incubating the plurality of captured truncated receptors with detection antibodies comprising one or a plurality of activation state-independent antibodies and one or a plurality of activation state-dependent antibodies specific for an ICD binding region of the full-length receptor to form a plurality of detectable captured truncated receptors,
wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair;
(v) incubating the plurality of detectable captured truncated receptors with a second member of the signal amplification pair to generate an amplified signal; and
(vi) detecting the amplified signal generated from the first and second members of the signal amplification pair.

In certain embodiments, the truncated receptor is p95HER2 and the full-length receptor is HER2. In certain other embodiments, the plurality of beads specific for an extracellular domain (ECD) binding region comprises a streptavidin-biotin pair, wherein the biotin is attached to the bead and the biotin is attached to an antibody (e.g., wherein the antibody is specific for the ECD binding region of the full-length receptor).

In alternative embodiments, the activation state-dependent antibodies can be labeled with a facilitating moiety and the activation state-independent antibodies can be labeled with a first member of a signal amplification pair.

As another non-limiting example, the three antibodies used in the proximity assay can comprise: (1) a capture antibody specific for a particular analyte of interest; (2) a first detection antibody which detects the total amount of the analyte (i.e., a first activation state-independent antibody); and (3) a second detection antibody which detects the total amount of the analyte (i.e., a second activation state-independent antibody). In preferred embodiments, the first and second activation state-independent antibodies recognize different (e.g., distinct) epitopes on the analyte.

In one particular embodiment, the proximity assay for detecting the expression level of an analyte of interest comprises:
(i) incubating the cellular extract with one or a plurality of dilution series of capture antibodies to form a plurality of captured analytes;
(ii) incubating the plurality of captured analytes with detection antibodies comprising one or a plurality of first and second activation state-independent antibodies specific for the corresponding analytes to form a plurality of detectable captured analytes,
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(iii) incubating the plurality of detectable captured analytes with a second member of the signal amplification pair to generate an amplified signal; and
(iv) detecting the amplified signal generated from the first and second members of the signal amplification pair.

In another particular embodiment, the proximity assay for detecting the expression level of an analyte of interest that is a truncated receptor comprises:
(i) incubating the cellular extract with a plurality of beads specific for an extracellular domain (ECD) binding region of a full-length receptor;
(ii) removing the plurality of beads from the cellular extract, thereby removing the full-length receptor to form a cellular extract devoid of the full-length receptor;
(iii) incubating the cellular extract devoid of the full-length receptor with one or a plurality of capture antibodies specific for an intracellular domain (ICD) binding region of the full-length receptor to form a plurality of captured truncated receptors;
(iv) incubating the plurality of captured truncated receptors with detection antibodies comprising one or a plurality of first and second activation state-independent antibodies specific for an ICD binding region of the full-length receptor to form a plurality of detectable captured truncated receptors,
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(v) incubating the plurality of detectable captured truncated receptors with a second member of the signal amplification pair to generate an amplified signal; and
(vi) detecting the amplified signal generated from the first and second members of the signal amplification pair.

In certain embodiments, the truncated receptor is p95HER2 and the full-length receptor is HER2. In certain other embodiments, the plurality of beads specific for an extracellular domain (ECD) binding region comprises a streptavidin-biotin pair, wherein the biotin is attached to the bead and the biotin is attached to an antibody (e.g., wherein the antibody is specific for the ECD binding region of the full-length receptor).

In alternative embodiments, the first activation state-independent antibodies can be labeled with a first member of a signal amplification pair and the second activation state-independent antibodies can be labeled with a facilitating moiety.

The proximity assays described herein are typically antibody-based arrays which comprise one or a plurality of different capture antibodies at a range of capture antibody concentrations that are coupled to the surface of a solid support in different addressable locations. Examples of suitable solid supports for use in the present invention are described above.

The capture antibodies, activation state-independent antibodies, and activation state-dependent antibodies are preferably selected to minimize competition between them with respect to analyte binding (i.e., all antibodies can simultaneously bind their corresponding signal transduction molecules).

In some embodiments, activation state-independent antibodies for detecting activation levels of one or more of the analytes or, alternatively, first activation state-independent antibodies for detecting expression levels of one or more of the analytes further comprise a detectable moiety. In such instances, the amount of the detectable moiety is correlative to the amount of one or more of the analytes in the cellular extract. Examples of detectable moieties include, but are not limited to, fluorescent labels, chemically reactive labels, enzyme labels, radioactive labels, and the like. Preferably, the detectable moiety is a fluorophore such as an Alexa Fluor® dye (e.g., Alexa Fluor® 647), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The detectable moiety can be coupled directly or indirectly to the activation state-independent antibodies using methods well-known in the art.

In certain instances, activation state-independent antibodies for detecting activation levels of one or more of the analytes or, alternatively, first activation state-independent antibodies for detecting expression levels of one or more of the analytes are directly labeled with the facilitating moiety. The facilitating moiety can be coupled to activation state-independent antibodies using methods well-known in the art. A suitable facilitating moiety for use in the present invention includes any molecule capable of generating an oxidizing agent which channels to (i.e., is directed to) and reacts with (i.e., binds, is bound by, or forms a complex with) another molecule in proximity (i.e., spatially near or close) to the facilitating moiety. Examples of facilitating moieties include, without limitation, enzymes such as glucose oxidase or any other enzyme that catalyzes an oxidation/reduction reaction involving molecular oxygen (O₂) as the electron acceptor, and photosensitizers such as methylene blue, rose bengal, porphyrins, squarate dyes, phthalocyanines, and the like. Non-limiting examples of oxidizing agents include hydrogen peroxide (H₂O₂), a singlet oxygen, and any other compound that transfers oxygen atoms or gains electrons in an oxidation/reduction reaction. Preferably, in the presence of a suitable substrate (e.g., glucose, light, etc.), the facilitating moiety (e.g., glucose oxidase, photosensitizer, etc.) generates an oxidizing agent (e.g., hydrogen peroxide (H₂O₂), single oxygen, etc.) which channels to and reacts with the first member of the signal amplification pair (e.g., horseradish peroxidase (HRP), hapten protected by a protecting group, an enzyme inactivated by thioether linkage to an enzyme inhibitor, etc.) when the two moieties are in proximity to each other.

In certain other instances, activation state-independent antibodies for detecting activation levels of one or more of the analytes or, alternatively, first activation state-independent antibodies for detecting expression levels of one or more of the analytes are indirectly labeled with the facilitating moiety via hybridization between an oligonucleotide linker conjugated to the activation state-independent antibodies and a complementary oligonucleotide linker conjugated to the facilitating moiety. The oligonucleotide linkers can be coupled to the facilitating moiety or to the activation state-independent antibodies using methods well-known in the art. In some embodiments, the oligonucleotide linker conjugated to the facilitating moiety has 100% complementarity to the oligonucleotide linker conjugated to the activation state-independent antibodies. In other embodiments, the oligonucleotide linker pair comprises at least one, two, three, four, five, six, or more mismatch regions, e.g., upon hybridization under stringent hybridization conditions. One skilled in the art will appreciate that activation state-independent antibodies specific for different analytes can either be conjugated to the same oligonucleotide linker or to different oligonucleotide linkers.

The length of the oligonucleotide linkers that are conjugated to the facilitating moiety or to the activation state-independent antibodies can vary. In general, the linker sequence can be at least about 5, 10, 15, 20, 25, 30, 35, 40, 45, 50, 75, or 100 nucleotides in length. Typically, random nucleic acid sequences are generated for coupling. As a non-limiting example, a library of oligonucleotide linkers can be designed to have three distinct contiguous domains: a spacer domain; signature domain; and conjugation domain. Preferably, the oligonucleotide linkers are designed for efficient coupling without destroying the function of the facilitating moiety or activation state-independent antibodies to which they are conjugated.

The oligonucleotide linker sequences can be designed to prevent or minimize any secondary structure formation under a variety of assay conditions. Melting temperatures are typically carefully monitored for each segment within the linker to allow their participation in the overall assay procedures. Generally, the range of melting temperatures of the segment of the linker sequence is between 1-10° C. Computer algorithms (e.g., OLIGO 6.0) for determining the melting temperature, secondary structure, and hairpin structure under defined ionic concentrations can be used to analyze each of the three different domains within each linker. The overall combined sequences can also be analyzed for their structural characterization and their comparability to other conjugated oligonucleotide linker sequences, e.g., whether they will hybridize under stringent hybridization conditions to a complementary oligonucleotide linker.

The spacer region of the oligonucleotide linker provides adequate separation of the conjugation domain from the oligonucleotide crosslinking site. The conjugation domain functions to link molecules labeled with a complementary oligonucleotide linker sequence to the conjugation domain via nucleic acid hybridization. The nucleic acid-mediated hybridization can be performed either before or after antibody-analyte (i.e., antigen) complex formation, providing a more flexible assay format. Unlike many direct antibody conjugation methods, linking relatively small oligonucleotides to antibodies or other molecules has minimal impact on the specific affinity of antibodies towards their target analyte or on the function of the conjugated molecules.

In some embodiments, the signature sequence domain of the oligonucleotide linker can be used in complex multiplexed protein assays. Multiple antibodies can be conjugated with oligonucleotide linkers with different signature sequences. In multiplex immunoassays, reporter oligonucleotide sequences labeled with appropriate probes can be used to detect cross-reactivity between antibodies and their antigens in the multiplex assay format.

Oligonucleotide linkers can be conjugated to antibodies or other molecules using several different methods. For example, oligonucleotide linkers can be synthesized with a thiol group on either the 5′ or 3′ end. The thiol group can be deprotected using reducing agents (e.g., TCEP-HCl) and the resulting linkers can be purified by using a desalting spin column. The resulting deprotected oligonucleotide linkers can be conjugated to the primary amines of antibodies or other types of proteins using heterobifunctional cross linkers such as SMCC. Alternatively, 5′-phosphate groups on oligonucleotides can be treated with water-soluble carbodiimide EDC to form phosphate esters and subsequently coupled to amine-containing molecules. In certain instances, the diol on the 3′-ribose residue can be oxidized to aldehyde groups and then conjugated to the amine groups of antibodies or other types of proteins using reductive amination. In certain other instances, the oligonucleotide linker can be synthesized with a biotin modification on either the 3′ or 5′ end and conjugated to streptavidin-labeled molecules.

Oligonucleotide linkers can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). In general, the synthesis of oligonucleotides makes use of common nucleic acid protecting and coupling groups, such as dimethoxytrityl at the 5′-end and phosphoramidites at the 3′-end. Suitable reagents for oligonucleotide synthesis, methods for nucleic acid deprotection, and methods for nucleic acid purification are known to those of skill in the art.

In certain instances, activation state-dependent antibodies for detecting activation levels of one or more of the analytes or, alternatively, second activation state-independent antibodies for detecting expression levels of one or more of the analytes are directly labeled with the first member of the signal amplification pair. The signal amplification pair member can be coupled to activation state-dependent antibodies to detect activation levels or second activation state-independent antibodies to detect expression levels using methods well-known in the art. In certain other instances, activation state-dependent antibodies or second activation state-independent antibodies are indirectly labeled with the first member of the signal amplification pair via binding between a first member of a binding pair conjugated to the activation state-dependent antibodies or second activation state-independent antibodies and a second member of the binding pair conjugated to the first member of the signal amplification pair. The binding pair members (e.g., biotin/streptavidin) can be coupled to the signal amplification pair member or to the activation state-dependent antibodies or second activation state-independent antibodies using methods well-known in the art. Examples of signal amplification pair members include, but are not limited to, peroxidases such horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, and the like. Other examples of signal amplification pair members include haptens protected by a protecting group and enzymes inactivated by thioether linkage to an enzyme inhibitor.

In one example of proximity channeling, the facilitating moiety is glucose oxidase (GO) and the first member of the signal amplification pair is horseradish peroxidase (HRP). When the GO is contacted with a substrate such as glucose, it generates an oxidizing agent (i.e., hydrogen peroxide (H₂O₂)). If the HRP is within channeling proximity to the GO, the H₂O₂ generated by the GO is channeled to and complexes with the HRP to form an HRP-H₂O₂ complex, which, in the presence of the second member of the signal amplification pair (e.g., a chemiluminescent substrate such as luminol or isoluminol or a fluorogenic substrate such as tyramide (e.g., biotin-tyramide), homovanillic acid, or 4-hydroxyphenyl acetic acid), generates an amplified signal. Methods of using GO and HRP in a proximity assay are described in, e.g., Langry et al., U.S. Dept. of Energy Report No. UCRL-ID-136797 (1999). When biotin-tyramide is used as the second member of the signal amplification pair, the HRP-H₂O₂ complex oxidizes the tyramide to generate a reactive tyramide radical that covalently binds nearby nucleophilic residues. The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor® dye (e.g., Alexa Fluor® 555), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS), 4-chloro-1-napthol (4CN), and/or porphyrinogen.

In another example of proximity channeling, the facilitating moiety is a photosensitizer and the first member of the signal amplification pair is a large molecule labeled with multiple haptens that are protected with protecting groups that prevent binding of the haptens to a specific binding partner (e.g., ligand, antibody, etc.). For example, the signal amplification pair member can be a dextran molecule labeled with protected biotin, coumarin, and/or fluorescein molecules. Suitable protecting groups include, but are not limited to, phenoxy-, analino-, olefin-, thioether-, and selenoether-protecting groups. Additional photosensitizers and protected hapten molecules suitable for use in the proximity assays of the present invention are described in U.S. Pat. No. 5,807,675. When the photosensitizer is excited with light, it generates an oxidizing agent (i.e., singlet oxygen). If the hapten molecules are within channeling proximity to the photosensitizer, the singlet oxygen generated by the photosensitizer is channeled to and reacts with thioethers on the protecting groups of the haptens to yield carbonyl groups (ketones or aldehydes) and sulphinic acid, releasing the protecting groups from the haptens. The unprotected haptens are then available to specifically bind to the second member of the signal amplification pair (e.g., a specific binding partner that can generate a detectable signal). For example, when the hapten is biotin, the specific binding partner can be an enzyme-labeled streptavidin. Exemplary enzymes include alkaline phosphatase, β-galactosidase, HRP, etc. After washing to remove unbound reagents, the detectable signal can be generated by adding a detectable (e.g., fluorescent, chemiluminescent, chromogenic, etc.) substrate of the enzyme and detected using suitable methods and instrumentation known in the art. Alternatively, the detectable signal can be amplified using tyramide signal amplification and the activated tyramide either directly detected or detected upon the addition of a signal-detecting reagent as described above.

In yet another example of proximity channeling, the facilitating moiety is a photosensitizer and the first member of the signal amplification pair is an enzyme-inhibitor complex. The enzyme and inhibitor (e.g., phosphonic acid-labeled dextran) are linked together by a cleavable linker (e.g., thioether). When the photosensitizer is excited with light, it generates an oxidizing agent (i.e., singlet oxygen). If the enzyme-inhibitor complex is within channeling proximity to the photosensitizer, the singlet oxygen generated by the photosensitizer is channeled to and reacts with the cleavable linker, releasing the inhibitor from the enzyme, thereby activating the enzyme. An enzyme substrate is added to generate a detectable signal, or alternatively, an amplification reagent is added to generate an amplified signal.

In a further example of proximity channeling, the facilitating moiety is HRP, the first member of the signal amplification pair is a protected hapten or an enzyme-inhibitor complex as described above, and the protecting groups comprise p-alkoxy phenol. The addition of phenylenediamine and H₂O₂ generates a reactive phenylene diimine which channels to the protected hapten or the enzyme-inhibitor complex and reacts with p-alkoxy phenol protecting groups to yield exposed haptens or a reactive enzyme. The amplified signal is generated and detected as described above (see, e.g., U.S. Pat. Nos. 5,532,138 and 5,445,944).

An exemplary protocol for performing the proximity assays described herein is provided in Example 4 of PCT Publication No. WO2009/108637, the disclosure of which is herein incorporated by reference in its entirety for all purposes.

In another embodiment, the present invention provides kits for performing the proximity assays described above comprising: (a) a dilution series of one or a plurality of capture antibodies restrained on a solid support; and (b) one or a plurality of detection antibodies (e.g., a combination of activation state-independent antibodies and activation state-dependent antibodies for detecting activation levels and/or a combination of first and second activation state-independent antibodies for detecting expression levels). In some instances, the kits can further contain instructions for methods of using the kit to detect the expression and/or activation status of one or a plurality of signal transduction molecules of cells such as tumor cells. The kits may also contain any of the additional reagents described above with respect to performing the specific methods of the present invention such as, for example, first and second members of the signal amplification pair, tyramide signal amplification reagents, substrates for the facilitating moiety, wash buffers, etc.

A. Detection Assays for Dimerization

In certain aspects, the present invention provides an assay for detecting and/or quantitating homo- or heterodimerization of receptor tyrosine kinases including, but not limited to, HER1:HER2 dimers, HER1:HER3 dimers, HER2:HER3 dimers, HER2:HER2 dimers, HER2:HER4 dimers, p95HER2:HER3 dimers, p95HER2:HER2 dimers, and the like. A homodimer is formed by two identical molecules such as HER2:HER2 in a process called homodimerization, whereas a heterodimer is formed by two different macromolecules such as HER1:HER3 or HER2:HER3 in a process called heterodimerization. In this aspect, the assay comprises three antibodies: (1) a capture antibody specific for one member of the dimer pair; (2) a first detection antibody specific for a first member of the dimer pair, wherein the first detection antibody is specific for a different domain than the capture antibody; and a (3) a second detection antibody specific for a second member of the dimer pair.

Certain of the CEER techniques are described above and disclosed in U.S. Pat. No. 8,163,299, U.S. Patent Publication Nos. 20080261829, 20090035792, 20100167945, 20110071042 and 20110281748. Further details can be found, e.g., in WO 2010/132723, WO 2011/008990, and WO 2013/033623, the disclosures of which are hereby incorporated by reference in their entireties.

In one embodiment of the foregoing proximity assay for detection of dimerization of receptor tyrosine kinases, a capture antibody is used to capture a member of the RTK dimer, for example, HER2. A first detection antibody is then used to bind to a different portion (e.g., epitope) on HER2. A second detection antibody is thereafter used to bind to the dimerized second receptor tyrosine kinase, for example, HER3. The first detection antibody comprises one or a plurality of first activation state-independent antibodies specific for one member of the dimer, whereas a second detection antibody or a plurality of second detection antibodies is specific for the other member of the dimer. The first detection antibody is labeled with a facilitating moiety, e.g., glucose oxidase (GO) and the second detection antibody is labeled with a first member of a signal amplification pair, e.g., horseradish peroxidase (HRP). The facilitating moiety generates an oxidizing agent, e.g., hydrogen peroxide, which channels to and reacts with the first member of the signal amplification pair. Thereafter, the plurality of detectable captured analytes are incubated with a second member of the signal amplification pair, e.g., tyramide or tyramide biotin to generate an amplified signal, which is then detected.

Suitable activation state-independent antibodies for measuring dimerization of receptor tyrosine kinases include any antibody that binds to an epitope on a receptor tyrosine kinase having an amino acid residue that has not been activated (e.g., phosphorylated). Activation state-independent antibodies that bind to RTKs such as members of the ErbB family, cMET, IGF-1R, and the like that are suitable for use in the present invention are commercially available from, but not limited to, Cell Signaling Technology (Danvers, Mass.), Thermo Scientific (Waltham, Mass.), Abcam (Cambridge, Mass.), Santa Cruz Biotechnology (Santa Cruz, Calif.), Sigma-Aldrich (St. Louis, Mo.), and EMD Millipore (Billerica, Mass.).

Suitable activation state-dependent antibodies for measuring dimerization of receptor tyrosine kinases include any antibody that binds to an epitope of a receptor tyrosine kinase having an amino acid residue that has been activated (e.g., phosphorylated). Activation state-dependent antibodies that bind to RTKs such as members of the ErbB family, cMET, IGF-1R, and the like that are suitable for use in the present invention are commercially available from, but not limited to, Cell Signaling Technology (Danvers, Mass.), Thermo Scientific (Waltham, Mass.), Abcam (Cambridge, Mass.), Santa Cruz Biotechnology (Santa Cruz, Calif.), Sigma-Aldrich (St. Louis, Mo.), and EMD Millipore (Billerica, Mass.).

In some embodiments, the assay method for detecting and/or quantitating homo- or heterodimerization of receptor tyrosine kinases (RTKs) comprises:

(a) measuring the dimerization of at least two receptor tyrosine kinases (RTKs), wherein measuring comprises: (i) incubating a cellular extract with one or a plurality of dilution series of capture antibodies to form a plurality of captured analytes; (ii) incubating the plurality of captured analytes with detection antibodies comprising a first or a plurality of first activation state-independent antibodies and a second or a plurality of second activation state-independent antibodies specific for a first member and a second member, respectively, of a dimerized pair of analytes to form a plurality of detectable captured dimerized analytes, wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair; (iii) incubating the plurality of detectable captured dimerized analytes with a second member of the signal amplification pair to generate an amplified signal; and (iv) detecting the amplified signal generated from the first and second members of the signal amplification pair; and

In certain instances, the dimerization of the at least two RTKs is compared to a reference dimerization profile of the same two RTKs, wherein the reference dimerization profile is optionally generated in the absence of an anticancer drug. In other instances, the method further comprises calibrating the level of dimerization of the at least two RTKs against a standard curve generated for the at least two RTKs.

In certain embodiments, the cellular extract is isolated from a cetuximab-sensitive subject with colorectal cancer. In other embodiments, the cellular extract is isolated from a subject with colorectal cancer receiving therapy (e.g., monotherapy) with cetuximab.

In some instances, the amount of amplified signal is correlative to the amount of dimerized receptor tyrosine kinase.

The capture antibodies and detection antibodies are preferably selected to minimize competition between them with respect to analyte binding (i.e., both capture and detection antibodies can simultaneously bind their corresponding signal transduction molecules).

A variety of facilitating moieties are useful in the present invention. A suitable facilitating moiety for use in the present invention includes any molecule capable of generating an oxidizing agent which channels to (i.e., is directed to) and reacts with (i.e., binds, is bound by, or forms a complex with) another molecule in proximity (i.e., spatially near or close) to the facilitating moiety. Examples of facilitating moieties include, without limitation, enzymes such as glucose oxidase or any other enzyme that catalyzes an oxidation/reduction reaction involving molecular oxygen (O₂) as the electron acceptor, and photosensitizers such as methylene blue, rose bengal, porphyrins, squarate dyes, phthalocyanines, and the like. Non-limiting examples of oxidizing agents include hydrogen peroxide (H₂O₂), a singlet oxygen, and any other compound that transfers oxygen atoms or gains electrons in an oxidation/reduction reaction. Preferably, in the presence of a suitable substrate (e.g., glucose, light, etc.), the facilitating moiety (e.g., glucose oxidase, photosensitizer, etc.) generates an oxidizing agent (e.g., hydrogen peroxide (H₂O₂), single oxygen, etc.) which channels to and reacts with the first member of the signal amplification pair (e.g., horseradish peroxidase (HRP), hapten protected by a protecting group, an enzyme inactivated by thioether linkage to an enzyme inhibitor, etc.) when the two moieties are in proximity to each other.

Suitable signal amplification pair members include, but are not limited to, peroxidases such horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, and the like. Other examples of signal amplification pair members include haptens protected by a protecting group and enzymes inactivated by thioether linkage to an enzyme inhibitor.

Non-limiting examples of proximity channeling suitable for detecting dimerization of receptors are described above and incorporated herein by reference in their entirety for all purposes. In one example of proximity channeling, the facilitating moiety is glucose oxidase (GO) and the first member of the signal amplification pair is horseradish peroxidase (HRP). In another example of proximity channeling, the facilitating moiety is a photosensitizer and the first member of the signal amplification pair is a large molecule labeled with multiple haptens that are protected with protecting groups that prevent binding of the haptens to a specific binding partner (e.g., ligand, antibody, etc.). In yet another example of proximity channeling, the facilitating moiety is a photosensitizer and the first member of the signal amplification pair is an enzyme-inhibitor complex. In yet a further example of proximity channeling, the facilitating moiety is HRP, the first member of the signal amplification pair is a protected hapten or an enzyme-inhibitor complex as described above, and the protecting groups comprise p-alkoxy phenol.

The methods of the invention are particularly useful for determining the presence or level of receptor dimerization (e.g., HER2/HER3 dimers) in cetuximab-sensitive subjects with colorectal cancer to select or identify subjects for combination therapy, to optimize therapy, to reduce toxicity, to monitor the efficacy of therapeutic treatment, and/or to detect adaptive non-responsivenes or resistance to therapy. In particular embodiments, combination therapy comprises an EGFR (ErbB1) inhibitor in combination with a HER2 (ErbB2) inhibitor.

B. Detection Assays for PI3K Complexes

The assays described herein can be used to detect and quantitate the amount of PI3K complex and the amount of activation and/or phosphorylation of a PI3K complex. The PI3K complex comprises: (i) a dimerized receptor tyrosine kinase pair; and (ii) a PI3K p85 subunit and a PI3K p110 (e.g., α or β) subunit. In particular embodiments, the assay comprises three antibodies: (1) a capture antibody specific for either the PI3K p85 or the PI3K p110 subunit; (2) a first detection antibody specific for a first member of the dimerized receptor tyrosine kinase pair or a PI3K subunit, wherein the first detection antibody is specific for a different domain than the capture antibody and wherein the PI3K subunit may be activated; and (3) a second detection antibody specific for a second member of the dimer pair or a PI3K subunit.

In some embodiments, a PI3K complex is detectable by the assays described herein as follows: (1) the PI3K p85 subunit is bound by the capture antibody; (2) a first detection antibody is specific for the PI3K p110 α or β subunit; and (3) a second detection antibody is specific for a first member of the dimer pair.

In other embodiments, an activated PI3K complex is detectable by the assays described herein as follows: (1) the PI3K p85 subunit is bound to the capture antibody; (2) a first detection antibody is specific for the PI3K p110 α or β subunit; and (3) a second detection antibody comprises an activation state-dependent antibody specific for a phosphorylation site on a PI3K subunit such as p85 (e.g., Y452, Y458, Y460, Y463, Y467, Y688, Y470, or other pTyr site).

In yet other embodiments, an activated PI3K complex is detectable by the assays described herein as follows: (1) the PI3K p85 subunit is bound by the capture antibody; (2) a first detection antibody comprises an activation state-independent antibody specific for a one member of a dimerized receptor tyrosine kinase (e.g., HER1, HER2, HER3, cMET, IGF-1R and the like); and (3) a second detection antibody comprises an activation state-dependent antibody specific for a phosphorylation site on a PI3K subunit such as p85 (e.g., Y452, Y458, Y460, Y463, Y467, Y688, Y470, or other pTyr site).

In still yet other embodiments, a PI3K complex is detectable by the assays described herein as follows: (1) the PI3K p85 subunit is bound by the capture antibody; (2) a first detection antibody comprises an activation state-independent antibody is specific for a one member of a dimerized receptor tyrosine kinase (e.g., HER1, HER2, HER3, cMET, IGF-1R, and the like); and (3) a second detection antibody comprises an activation state-independent antibody specific for the other member of the dimerized pair.

In some embodiments, the detection of PI3K complexes will also correlate with the detection of activated (e.g., phosphorylated) PI3K.

In further embodiments, a PI3K complex is detectable by the assays described herein as follows: (1) the PI3K p110 subunit is bound by the capture antibody; (2) a first detection antibody comprises an activation state-independent antibody specific for a one member of a dimerized receptor tyrosine kinase (e.g., HER1, HER2, HER3, cMET, IGF-1R, and the like); and (3) a second detection antibody comprises an activation state-dependent antibody specific for a phosphorylation site on a PI3K subunit such as p85 (e.g., Y452, Y458, Y460, Y463, Y467, Y688, Y470, or other pTyr site).

In yet further embodiments, a PI3K complex is detectable by the assays described herein as follows: (1) the PI3K p85 subunit is bound by the capture antibody; (2) a first detection antibody comprises an activation state-independent antibody specific for one member of a dimer of a receptor tyrosine kinase (e.g., HER1, HER2, HER3, cMET, IGF-1R, and the like); and (3) a second detection antibody comprises an activation state-dependent antibody specific for a phosphorylation site on a PI3K subunit such as p85 (e.g., Y452, Y458, Y460, Y463, Y467, Y688, Y470, or other pTyr site).

Suitable antibodies for measuring the level of a PI3K complex include any antibody that is specific for (i.e., recognizes, binds to, or forms a complex with) an epitope of the PI3K p110 subunit (e.g., α or β), the PI3K p85 subunit, or the dimerized receptor tyrosine kinase pair.

Suitable activation state-independent antibodies bind to an epitope of the PI3K p110 subunit, the PI3K p85 subunit or the dimerized receptor tyrosine kinase pair, wherein the epitope is free of phosphorylated amino acid residues. Such activation state-independent antibodies include PI3K p85 subunit antibodies (Cat. #4257, #4292 from Cell Signaling Technology; Cat. Nos. sc-12929, sc-56934, sc-56938, sc-71892, sc-71891, and sc-376112, sc-292114, and sc-131325 from Santa Cruz Biotechnology; Cat. Nos. ab86714, ab22653, ab40755, ab250, ab135253, ab71925, ab63040, ab90578, ab133595, ab135952, ab65261, and ab71522 from Abcam), PI3K p110 α subunit antibodies (Cat. #4249 and #4249 from Cell Signaling Technology; Cat. Nos. sc-7248, sc-7189, sc-8010,sc-7174, sc-1332, sc-1331), and PI3K p110 β subunit antibodies (Cat. #3011 from Cell Signaling Technology; Cat. Nos. sc-7248, sc-7189, sc-8010, sc-376641, sc-376412, sc-376492, sc-603, sc-7175, sc-602 from Santa Cruz Biotechnology; Cat. Nos. ab32569, ab55593, ab97322, and ab32874 from Abcam).

Suitable activation state-independent antibodies specific for dimerized RTKs include antibodies to HER1 (Cat. #2646, #2239, #2239, #2963, #3265, and #2232 from Cell Signaling Technology; Cat. Nos. sc-374607, sc-365829, sc-80543, sc-120, sc-03, sc-101, sc-373476, sc-31155, sc-71031, sc-81451 and sc-71037 from Santa Cruz Biotechnology), antibodies to HER2 (Cat. #2165, #2248, #3250 and #2242 from Cell Signaling Technology), antibodies to HER3 (Cat. #4754 from Cell Signaling Technology; Cat. Nos. sc-415, sc-7390, sc-292557, sc-81455, sc-81454, sc-71067, sc-53279, and sc-285 from Santa Cruz Biotechnology), and antibodies to HER4 (Cat. #4795 from Cell Signaling Technology; Cat. Nos. sc-31150, sc-8050, sc-81456, sc-71071, sc-71070, sc-53280, sc31151, sc-283, and sc-31149 from Santa Cruz Biotechnology).

In some embodiments, an antibody that binds to the PI3K p110 α subunit is used in the assays of the present invention. In some embodiments, an antibody that binds to the PI3K p110 β subunit is used in the assays of the present invention. Suitable activation-dependent antibodies against PI3K are described in U.S. Patent Publication No. 20080014595, the disclosure of which is hereby incorporated by reference in its entirety for all purposes. Antibodies to PI3K are also commercially available from, but not limited to, Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), BD Biosciences (San Jose, Calif.), Thermo Scientific (Waltham, Mass.), Abcam (Cambridge, Mass.), Sigma-Aldrich (St. Louis, Mo.), and EMD Millipore (Billerica, Mass.).

As a non-limiting example, suitable activation state-dependent antibodies bind to an epitope on the PI3K p110 subunit or the PI3K p85 subunit, wherein the epitope has at least one phosphorylated amino acid residue (e.g., pTyr). Such activation state-dependent antibodies include a p-PI3K p85 (Tyr458)/p55 (Tyr199) antibody (Cat. #4228 from Cell Signaling Technology), a p-PI3K p85 (Tyr67) antibody (Cat. # sc-293115 from Santa Cruz Biotechnology), and a p-PI3K p85 (Tyr607) antibody (Cat. No. ab61801 from Abcam). Phospho-PI3K p85 antibodies useful in the present invention are described in U.S. Patent Publication. No. 20080014595, the disclosure of which is hereby incorporated by reference in its entirety for all purposes. Likewise, PI3K p110 antibodies useful in the present invention are described in U.S. Pat. No. 6,274,327, the disclosure of which is hereby incorporated by reference in its entirety for all purposes.

In some embodiments, antibodies specific to PI3K antigens or fragments thereof can be used in the methods for measuring PI3K complexation.

Suitable activation state-dependent antibodies for measuring dimerization of RTKs include any antibody that binds to an epitope of a receptor tyrosine kinase having an amino acid residue that has been activated (e.g., phosphorylated). Activation state-dependent antibodies that bind to RTKs such as members of the ErbB family, cMET, IGF-1R, and the like that are suitable for use in the present invention are commercially available from but not limited to Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), BD Biosciences (San Jose, Calif.), Thermo Scientific (Waltham, Mass.), Abcam (Cambridge, Mass.), Sigma-Aldrich (St. Louis, Mo.), and EMD Millipore (Billerica, Mass.).

For example, suitable activation state-dependent antibodies specific for dimerized RTKs include antibodies to HER1 (Cat. #8808, #3056, #6963, #2231, #2641, #2235, #2237, #2238, #2236, #2234, #2220, #4404, and #4407 from Cell Signaling Technology; Cat. Nos. sc-16802, sc-12351, sc-16804, sc-16803, sc-101665, sc101668, sc-101667, and sc-101669 from Santa Cruz Biotechnology), antibodies to HER2 (Cat. #2244, #2241, #6942, #2249, and #2247 from Cell Signaling Technology),), antibodies to HER3 (Cat. #4561, #4787,#4791, #2842 and #8017 from Cell Signaling Technology; Cat. No. sc-135654 from Santa Cruz Biotechnology), and antibodies to HER4 (Cat. #3790 and #4757 from Cell Signaling Technology; Cat. Nos. sc-33040 and sc-81491 from Santa Cruz Biotechnology).

In one particular embodiment, the proximity assay for measuring (e.g., detecting and quantitating) the level of a PI3K complex, wherein the PI3K complex comprises (a) a dimerized receptor tyrosine kinase pair; (b) a PI3K p85 subunit and a PI3K p110 subunit, comprises:
(i) incubating a cellular extract with one or a plurality of dilution series of capture antibodies to form a plurality of captured analytes;
(ii) incubating the plurality of captured analytes with first detection antibodies comprising a first or a plurality of first activation state-independent antibodies specific for either one member of a dimerized receptor tyrosine kinase pair or a PI3K p110 subunit; and second detection antibodies comprising (a) a second or a plurality of second activation state-independent antibodies specific for either one member of a dimerized receptor tyrosine kinase pair, a PI3K p85 or a PI3K p110 subunit or (b) a second or a plurality of second activation state-dependent antibodies specific for a PI3K p85 subunit and/or a PI3K p110 subunit, to form a plurality of detectable captured dimerized and complexed analytes,
wherein the first detection antibodies are labeled with a facilitating moiety, the second detection antibodies are labeled with a first member of a signal amplification pair, and the facilitating moiety generates an oxidizing agent which channels to and reacts with the first member of the signal amplification pair;
(iii) incubating the plurality of detectable captured dimerized analytes with a second member of the signal amplification pair to generate an amplified signal; and
(iv) detecting the amplified signal generated from the first and second members of the signal amplification pair.

In certain instances, the level of the PI3K complex activation is compared to a reference PI3K complex activation profile, wherein the reference PI3K complex profile is optionally generated in the absence of an anticancer drug. In other instances, the level of PI3K complex is calibrated against a standard curve generated for the PI3K complex. In further instances, the amount of amplified signal is correlative to the amount of the PI3K complex.

In certain embodiments, the cellular extract is isolated from a cetuximab-sensitive subject with colorectal cancer. In other embodiments, the cellular extract is isolated from a subject with colorectal cancer receiving therapy (e.g., monotherapy) with cetuximab.

In some embodiments, the level of PI3K complex activation is determined by (a) comparing the amount of phospho-PI3K to the total level of PI3K present in the sample, and (b) establishing a ratio of activated PI3K complex to total PI3K. In some instances, the level of the PI3K complex activation is determined based on the ratio. In some instances, the level of the PI3K complex activation is below a cut-off threshold. In other instances, the level of the PI3K complex activation is above the cut-off threshold.

In certain embodiments, at least two RTKs is selected form the group consisting of a HER1/HER2 dimer, a HER1/HER3 dimer, a HER2/HER3 dimer, a HER2/HER2 dimer, a HER2/HER4 dimer, a p95HER2/HER3 dimer, and a p95HER2/HER2 dimer.

The capture antibodies and detection antibodies are preferably selected to minimize competition between them with respect to analyte binding (i.e., both capture and detection antibodies can simultaneously bind their corresponding signal transduction molecules).

A variety of facilitating moieties are useful in the present invention. A suitable facilitating moiety for use in the present invention includes any molecule capable of generating an oxidizing agent which channels to (i.e., is directed to) and reacts with (i.e., binds, is bound by, or forms a complex with) another molecule in proximity (i.e., spatially near or close) to the facilitating moiety. Examples of facilitating moieties include, without limitation, enzymes such as glucose oxidase or any other enzyme that catalyzes an oxidation/reduction reaction involving molecular oxygen (O₂) as the electron acceptor, and photosensitizers such as methylene blue, rose bengal, porphyrins, squarate dyes, phthalocyanines, and the like. Non-limiting examples of oxidizing agents include hydrogen peroxide (H₂O₂), a singlet oxygen, and any other compound that transfers oxygen atoms or gains electrons in an oxidation/reduction reaction. Preferably, in the presence of a suitable substrate (e.g., glucose, light, etc.), the facilitating moiety (e.g., glucose oxidase, photosensitizer, etc.) generates an oxidizing agent (e.g., hydrogen peroxide (H₂O₂), single oxygen, etc.) which channels to and reacts with the first member of the signal amplification pair (e.g., horseradish peroxidase (HRP), hapten protected by a protecting group, an enzyme inactivated by thioether linkage to an enzyme inhibitor, etc.) when the two moieties are in proximity to each other.

Suitable signal amplification pair members include, but are not limited to, peroxidases such horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, and the like. Other examples of signal amplification pair members include haptens protected by a protecting group and enzymes inactivated by thioether linkage to an enzyme inhibitor.

Non-limiting examples of proximity channeling suitable for detecting dimerization of receptors are described above and incorporated herein by reference in their entirety for all purposes. In one example of proximity channeling, the facilitating moiety is glucose oxidase (GO) and the first member of the signal amplification pair is horseradish peroxidase (HRP). In another example of proximity channeling, the facilitating moiety is a photosensitizer and the first member of the signal amplification pair is a large molecule labeled with multiple haptens that are protected with protecting groups that prevent binding of the haptens to a specific binding partner (e.g., ligand, antibody, etc.). In yet another example of proximity channeling, the facilitating moiety is a photosensitizer and the first member of the signal amplification pair is an enzyme-inhibitor complex. In yet a further example of proximity channeling, the facilitating moiety is HRP, the first member of the signal amplification pair is a protected hapten or an enzyme-inhibitor complex as described above, and the protecting groups comprise p-alkoxy phenol.

The methods of the invention are particularly useful for determining the presence or level of PI3K complex activation (e.g., phosphorylation) in cetuximab-sensitive subjects with colorectal cancer to select or identify subjects for combination therapy, to optimize therapy, to reduce toxicity, to monitor the efficacy of therapeutic treatment, and/or to detect adaptive non-responsivenes or resistance to therapy. In certain instances, the activation of the PI3K complex comprises one or more activated RTKs (e.g., HER1, HER2, HER3, p95HER2, cMET, and IGF-1R), a PI3K p85 subunit, and a PI3K p110 subunit. In particular instances, the combination therapy comprises an EGFR (ErbB1) inhibitor in combination with a HER2 (ErbB2) inhibitor.

### VI. Production of Antibodies

The generation and selection of antibodies not already commercially available for analyzing the levels of expression and activation of signal transduction molecules in tumor cells in accordance with the immunoassays of the present invention can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic (e.g., retain the functional binding regions of) antibodies can also be prepared from genetic information by various procedures. See, e.g., Antibody Engineering: A Practical Approach, Borrebaeck, Ed., Oxford University Press, Oxford (1995); and Huse et al., J. Immunol., 149:3914-3920 (1992).

Those skilled in the art will recognize that many approaches can be taken in producing antibodies or binding fragments and screening and selecting for affinity and specificity for the various polypeptides of interest, but these approaches do not change the scope of the present invention.

A more detailed description of polyclonal antibodies, monoclonal antibodies, humanized antibodies, human antibodies, bispecific antibodies, fragments thereof, and methods of purifying antibodies is found in PCT Publication No. WO 2010/132723, the disclosure of which is herein incorporated by reference in its entirety for all purposes.

### VII. Methods of Administration

According to the methods of the present invention, the anticancer drugs described herein are administered to a subject by any convenient means known in the art. One skilled in the art will appreciate that the EGFR and HER2 inhibitor therapy described herein can be administered as part of a combined therapeutic approach with other therapies such as, e.g., chemotherapy, radiotherapy, hormonal therapy, immunotherapy, and/or surgery.

Anticancer drugs can be administered with a suitable pharmaceutical excipient as necessary and can be carried out via any of the accepted modes of administration. Thus, administration can be, for example, oral, buccal, sublingual, gingival, palatal, intravenous, topical, subcutaneous, transcutaneous, transdermal, intramuscular, intra joint, parenteral, intra-arteriole, intradermal, intraventricular, intracranial, intraperitoneal, intravesical, intrathecal, intralesional, intranasal, rectal, vaginal, or by inhalation. By “co-administer” it is meant that an anticancer drug is administered at the same time, just prior to, or just after the administration of a second drug (e.g., another anticancer drug in the combination therapy).

A therapeutically effective amount of an anticancer drug may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for human subjects and other mammals, each unit containing a predetermined quantity of an anticancer drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the anticancer drug.

Methods for preparing such dosage forms are known to those skilled in the art (see, e.g., REMINGTON'S PHARMACEUTICAL SCIENCES, 18TH ED., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with an anticancer drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An anticancer drug can also be formulated into a suppository disposed, for example, in a polyethylene glycol (PEG) carrier.

Liquid dosage forms can be prepared by dissolving or dispersing an anticancer drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. An anticancer drug can also be formulated into a retention enema.

For topical administration, the therapeutically effective dose can be in the form of emulsions, lotions, gels, foams, creams, jellies, solutions, suspensions, ointments, and transdermal patches. For administration by inhalation, an anticancer drug can be delivered as a dry powder or in liquid form via a nebulizer. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to a subject.

A subject can also be monitored at periodic time intervals to assess the efficacy of a certain therapeutic regimen. For example, the expression levels or activation states of certain signal transduction molecules or complexes thereof may change based on the therapeutic effect of treatment with one or more of the anticancer drugs described herein. The subject can be monitored to assess response and understand the effects of certain drugs or treatments in an individualized approach. Additionally, subjects who initially respond to a specific anticancer drug or combination of anticancer drugs may become refractory to the drug or drug combination, indicating that these subjects have developed acquired drug resistance. These subjects can be discontinued on their current therapy and an alternative treatment prescribed in accordance with the methods of the invention, such as, e.g., combination therapy with EGFR and HER2 inhibitors or therapy with a dual EGFR/HER2 inhibitor.

### VIII. Example

The following example is offered to illustrate, but not to limit, the claimed invention.

### Example 1

### EGFR Inhibition Leads to HER3/PI3K Activation by Feedback Induction of ErbB Heterodimers in Cetuximab-Sensitive Colon Cancer Cells

Even though cetuximab treatment has been successful for the treatment of KRAS wild-type colorectal cancers, complete remissions are rarely seen in patients, leading ultimately to resistance due to acquired mutations. We hypothesized that, even in cetuximab-sensitive patients, the ErbB network is insufficiently targeted since HER network plasticity may occur by relief of feedback signaling.

### Methods

We have used EGFR-sensitive colorectal cancer cells and investigated ErbB network activity and adaptations by RTK arrays, Western blot and Collaborative Enzyme Enhance Reactive ImmunoAssay (CEER). These were complemented by ErbB heterodimerization assays using CEER. Effects on cell survival were measured using colony formation and cell viability assays. mRNA expression of EGFR ligands EREG and AREG was performed using q-RT-PCR.

### Results

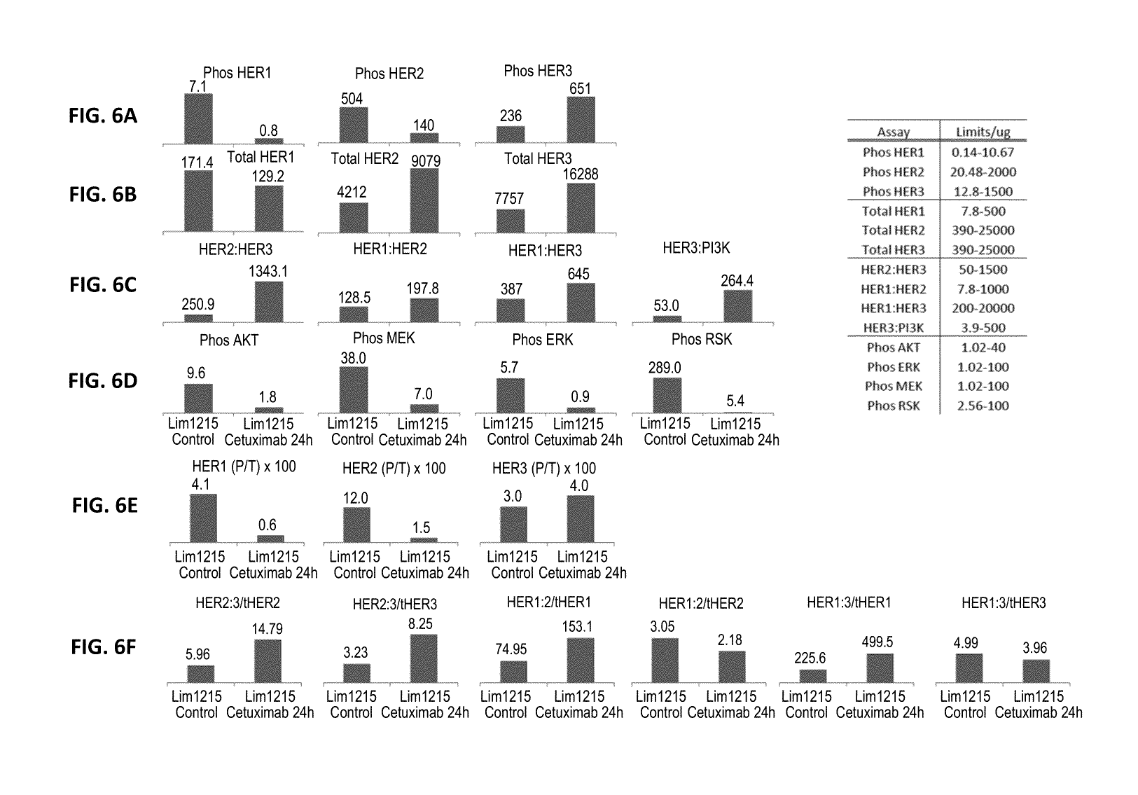

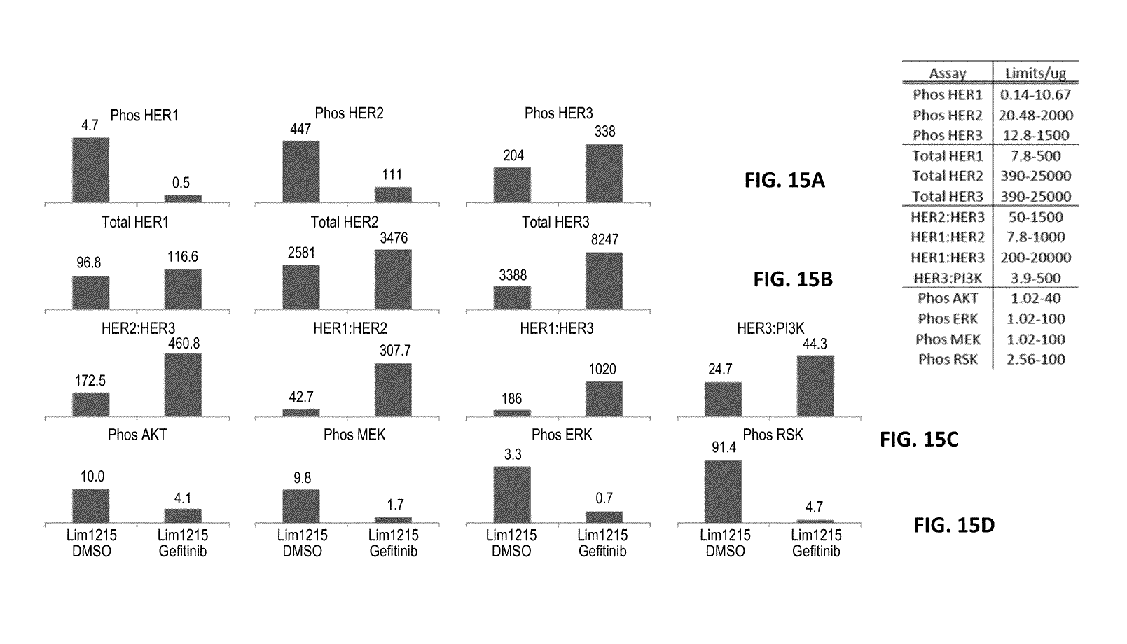

While EGFR was potently inhibited by both cetuximab and gefitinib, we observed a steady increase in HER2 and HER3 protein levels up to 24 h of treatment of EGFR sensitive colorectal cancer cell lines with cetuximab or gefitinib. See, for example, FIG. 6B and FIG. 15B.

While EGFR, Akt and Erk phosphorylation were potently inhibited, this association was linked with increased HER3 phosphorylation. See FIGS. 6A and 6D, as well as FIGS. 15A and 15D.

Concurrent with these results, we observe increased ErbB heterodimer formation upon EGFR inhibition in these cells. This heterodimer induction was accompanied by increased PI3K binding to HER3, resulting in enhanced HER3 signaling. See FIGS. 6C and 15C.

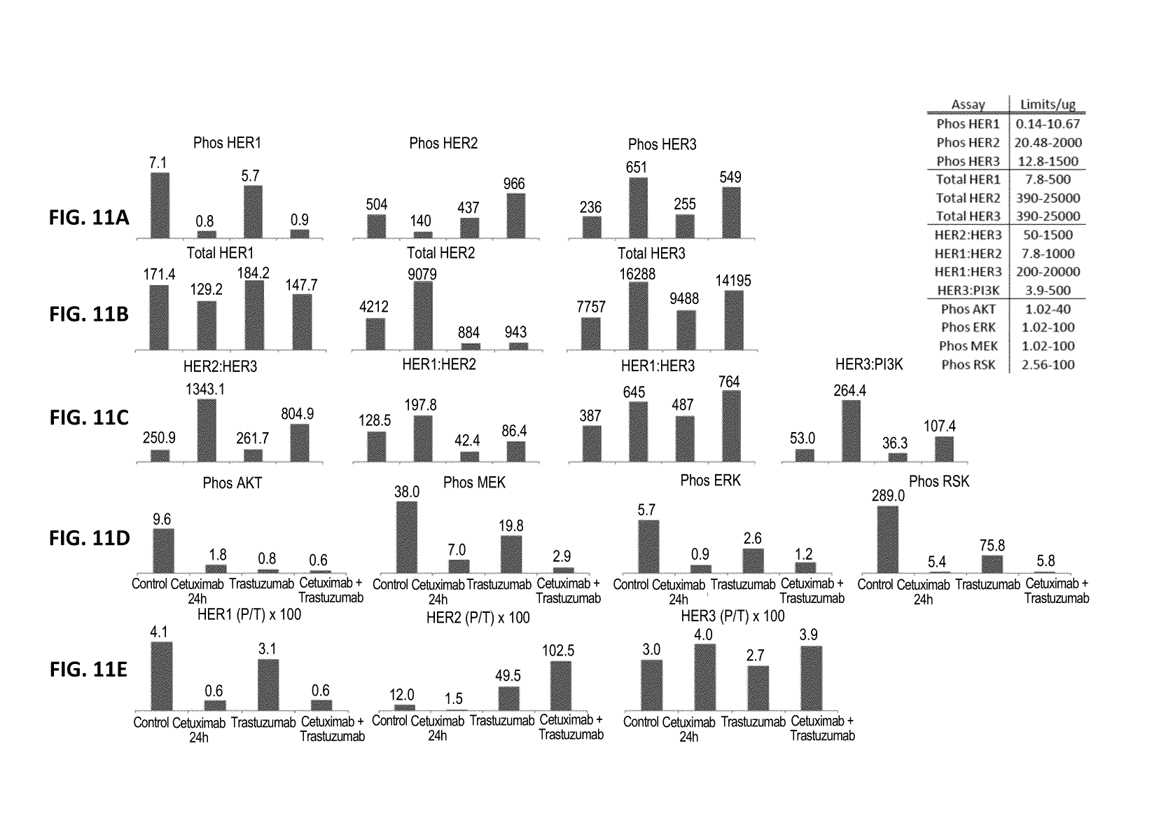

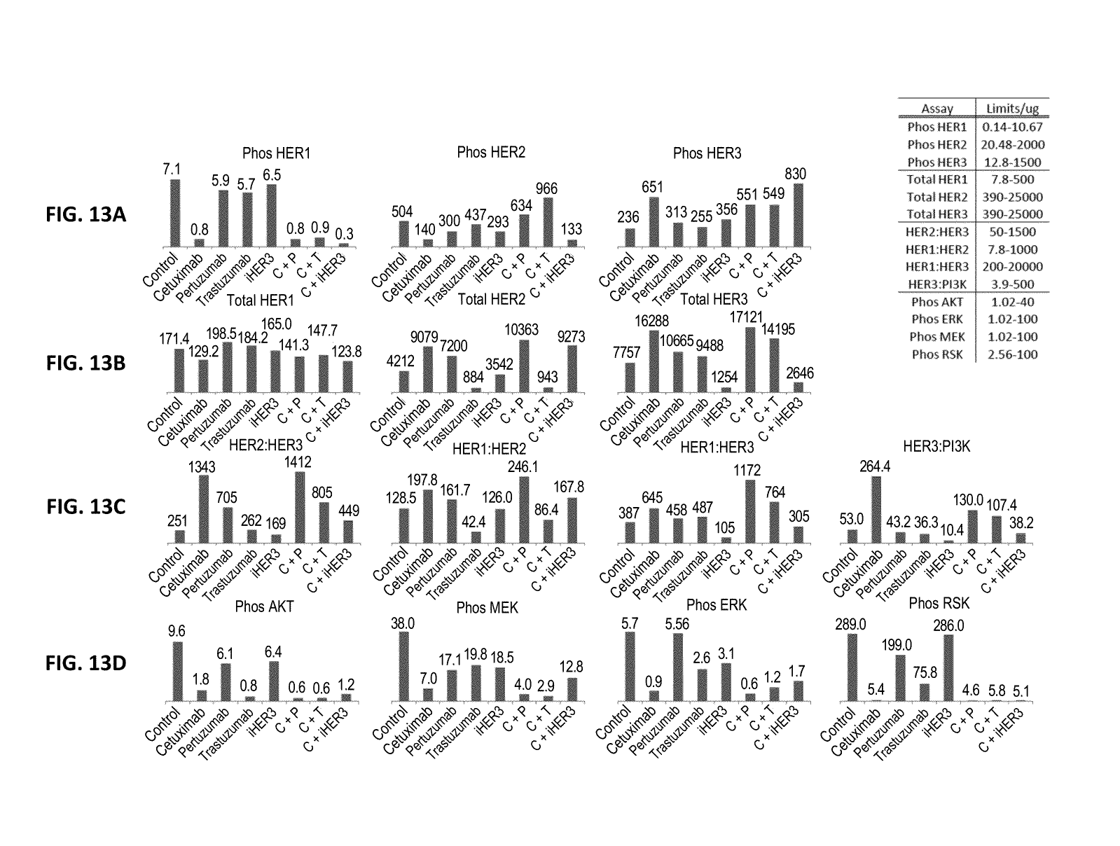

We next explored whether co-treatment of these cells with the HER2 inhibitor trastuzumab and lapatinib could rescue the feedback activation of HER3 in this context. In fact, co-treatment with cetuximab and trastuzumab blocked the induction of HER2-HER3 dimers induced by cetuximab, and blocked HER3 phosphorylation and HER3-PI3K binding. See, for example, FIG. 11A-C. Similar effects were observed for pHER3 and the HER3-PI3K interaction by treatment with the dual EGFR/HER2 inhibitor lapatinib. See FIG. 13A-C. Data for treatment of cells with cetuximab, pertuzumab, trastuzumab, a HER3 inhibitor, gefitinib, lapatinib, a MEK inhibitor, and various combinations thereof is summarized in Tables 1-3 below.

TABLE 1
ErbB Dimer Formation

| | HER2:HER3 | HER1:HER2 | HER1:HER3 | HER3:PI3K |
|---|---|---|---|---|
| | Assay Range | | | |
| | 50-1500 | 7.8-1000 | 200-20000 | 3.9-500 |
| | HER2:3 | HER1:2 | HER1:3 | HER3:PI3K |
| | (2 ug) | (2 ug) | (0.5 ug) | (4 ug) |
| Cetuximab 0 h | 89.8 | 46.2 | 121 | 21.4 |
| | | | (<LLOQ) | |
| Cetuximab 30 min | 81.6 | 36.9 | 128 | 57.3 |
| | | | (<LLOQ) | |
| Cetuximab 1 h | 101.1 | 49.3 | 107 | 35.2 |
| | | | (<LLOQ) | |
| Cetuximab 2 h | 80.9 | 35.4 | 89 | 46.2 |
| | | | (<LLOQ) | |
| Cetuximab4 h | 73.5 | 34.5 | 116 | 39.0 |
| | | | (<LLOQ) | |
| Cetuximab 6 h | 126.6 | 38.0 | 147 | 53.0 |
| | | | (<LLOQ) | |
| Cetuximab 8 h | 112.2 | 36.7 | 129 | 39.4 |
| | | | (<LLOQ) | |
| Control | 250.9 | 128.5 | 387 | 53.0 |
| Cetuximab 24 h | 1343.1 | 197.8 | 645 | 264.4 |
| Pertuzumab 24 h | 704.6 | 161.7 | 458 | 43.2 |
| Cetuximab + Pertuzumab | 1411.9 | 246.1 | 1172 | 130.0 |
| Trastuzumab | 261.7 | 42.4 | 487 | 36.3 |
| Cetuximab + Trastuzumab | 804.9 | 86.4 | 764 | 107.4 |
| iHER3 | 169.2 | 126.0 | 105 | 10.4 |
| | | | (<LLOQ) | |
| Cetuximab + iHER3 | 449.5 | 167.8 | 305 | 38.2 |
| control | 211.8 | 46.0 | 398 | 57.2 |
| Cetuximab 24 h | 952.8 | 79.5 | 1090 | 122.0 |
| Pertuzumab | 472.1 | 64.8 | 412 | 49.5 |
| Cetuximab + Pertuzumab | 744.3 | 135.0 | 561 | 97.3 |
| Trastuzumab | 290.9 | 30.6 | 374 | 37.7 |
| Cetuximab + Trastuzumab | 648.2 | 78.4 | 1162 | 145.4 |
| Gefitinib | 1167.6 | 454.5 | 5069 | 185.4 |
| Lapatinib | 493.6 | 63.6 | 2015 | 115.7 |
| DMSO | 172.5 | 42.7 | 186 | 24.7 |
| Cetuximab + DMSO | 434.0 | 58.3 | 353 | 173.7 |
| Gefitinib | 460.8 | 307.7 | 1020 | 44.3 |
| Lapatinib | 301.3 | 29.6 | 459 | 23.0 |
| iMEK AS703026 | 313.2 | 52.1 | 226 | 27.8 |

TABLE 2
ErbB Expression and Activation Levels

| | Total | Total | Total | Phos | Phos | Phos |
|---|---|---|---|---|---|---|
| | HER1 | HER2 | HER3 | HER1 | HER2 | HER3 |
| Assay Range | 7.8- | 390- | 390- | 0.14- | 20.48- | 12.8- |
| | 500 | 25000 | 25000 | 10.67 | 2000 | 1500 |
| | Total | | | Phospho | | |
| | HER1 | HER2 | HER3 | HER1 | HER2 | HER3 |
| | (1 ug) | (0.2 ug) | (0.2 ug) | (10 ug) | (2.5 ug) | (10 ug) |
| Cetuximab 0 h | 74.4 | 2127 | 3882 | 1.8 | 346 | 257 |
| Cetuximab 30 min | 40.9 | 2532 | 4115 | 0.3 | 105 | 223 |
| Cetuximab 1 h | 67.4 | 2853 | 5073 | 0.4 | 95 | 263 |
| Cetuximab 2 h | 46.9 | 2625 | 4707 | 0.5 | 86 | 261 |
| Cetuximab 4 h | 52.8 | 2529 | 5219 | 0.5 | 101 | 264 |
| Cetuximab 6 h | 60.2 | 2229 | 4976 | 0.4 | 129 | 200 |
| Cetuximab 8 h | 14.4 | 3109 | 5360 | 0.4 | 163 | 273 |
| Control | 171.4 | 4212 | 7757 | 7.1 | 504 | 236 |
| Cetuximab 24 h | 129.2 | 9079 | 16288 | 0.8 | 140 | 651 |
| Pertuzumab 24 h | 198.5 | 7200 | 10665 | 5.9 | 300 | 313 |
| Cetuximab + | 141.3 | 10363 | 17121 | 0.8 | 634 | 551 |
| Pertuzumab | | | | | | |
| Trastuzumab | 184.2 | 884 | 9488 | 5.7 | 437 | 255 |
| Cetuximab + | 147.7 | 943 | 14195 | 0.9 | 966 | 549 |
| Trastuzumab | | | | | | |
| iHER3 | 165.0 | 3542 | 1254 | 6.5 | 293 | 356 |
| Cetuximab + iHER3 | 123.8 | 9273 | 2646 | 0.3 | 133 | 830 |
| control | 137.2 | 3025 | 4282 | 7.4 | 2068 | 573 |
| | | | | | (>ULOQ) | |
| Cetuximab 24 h | 86.9 | 5678 | 8514 | 3.0 | 1547 | 1450 |
| Pertuzumab | 115.1 | 5568 | 5501 | 4.0 | 3187 | 826 |
| | | | | | (>ULOQ) | |
| Cetuximab + | 79.8 | 5929 | 4843 | 2.0 | 1908 | 1212 |
| Pertuzumab | | | | | | |
| Trastuzumab | 148.4 | 592 | 3743 | 6.4 | 2388 | 754 |
| | | | | | (>ULOQ) | |
| Cetuximab + | 88.6 | 839 | 11236 | 1.2 | 1843 | 1417 |
| Trastuzumab | | | | | | |
| Gefitinib | 172.9 | 5980 | 9789 | 1.5 | 544 | 954 |
| Lapatinib | 140.3 | 5391 | 5711 | 1.2 | 49 | 677 |
| DMSO | 96.8 | 2581 | 3388 | 4.7 | 447 | 204 |
| Cetuximab + DMSO | 46.5 | 4147 | 7789 | 0.7 | 385 | 637 |
| Gefitinib | 116.6 | 3476 | 8247 | 0.5 | 111 | 338 |
| Lapatinib | 98.2 | 4135 | 6907 | 0.4 | 15 | 234 |
| | | | | | (<LLOQ) | |
| iMEK AS703026 | 69.9 | 2546 | 3317 | 2.9 | 480 | 300 |

TABLE 3
Expression and Activation Levels of Signal Cascade Members

| | Phos AKT | Phos ERK | Phos MEK | Phos RSK |
|---|---|---|---|---|
| | Assay Range | | | |
| | 1.02-40 | 1.02-100 | 1.02-100 | 2.56-100 |
| | Phospho (pg/ug) | | | |
| | AKT (10 ug) | ERK (10 ug) | MEK (10 ug) | RSK (10 ug) |
| Cetuximab 0 h | 3.5 | 3.3 | 16.6 | 76.0 |
| Cetuximab 30 min | 0.92 | 1.1 | 9.0 | 46.8 |
| | (<LLOQ) | | | |
| Cetuximab 1 h | 3.6 | 1.9 | 7.3 | 45.7 |
| Cetuximab 2 h | 1.8 | 1.4 | 7.0 | 22.9 |
| Cetuximab4 h | 1.3 | 1.1 | 3.4 | 10.9 |
| Cetuximab 6 h | 1.7 | 0.56 | 2.9 | 8.4 |
| | | (<LLOQ) | | |
| Cetuximab 8 h | 1.7 | 0.7 | 2.5 | 5.2 |
| | | (<LLOQ) | | |
| Control | 9.6 | 5.7 | 38.0 | 289 |
| | | | | (>ULOQ) |
| Cetuximab 24 h | 1.8 | 0.87 | 7.0 | 5.4 |
| | | (<LLOQ) | | |
| Pertuzumab 24 h | 6.1 | 5.6 | 17.1 | 199 |
| | | | | (>ULOQ) |
| Cetuximab + Pertuzumab | 0.55 | 0.64 | 4.0 | 4.6 |
| | (<LLOQ) | (<LLOQ) | | |
| Trastuzumab | 0.79 | 2.6 | 19.8 | 75.8 |
| | (<LLOQ) | | | |
| Cetuximab + Trastuzumab | 0.56 | 1.2 | 2.9 | 5.8 |
| | (<LLOQ) | | | |
| iHER3 | 6.4 | 3.1 | 18.5 | 286 |
| | | | | (>ULOQ) |
| Cetuximab + iHER3 | 1.2 | 1.7 | 12.8 | 5.1 |
| control | 11.4 | 5.3 | 16.3 | 122 |
| | | | | (>ULOQ) |
| Cetuximab 24 h | 1.0 | 1.1 | 2.7 | 7.1 |
| Pertuzumab | 1.2 | 2.2 | 8.6 | 92.1 |
| Cetuximab + Pertuzumab | 0.47 | 1.3 | 2.2 | 5.1 |
| | (<LLOQ) | | | |
| Trastuzumab | 2.6 | 3.6 | 9.0 | 60.2 |
| Cetuximab + Trastuzumab | 0.31 | 1.1 | 3.7 | 4.2 |
| | (<LLOQ) | | | |
| Gefitinib | 0.64 | 1.1 | 2.9 | 6.3 |
| | (<LLOQ) | | | |
| Lapatinib | 0.66 | 0.84 | 3.1 | 3.9 |
| | (<LLOQ) | (<LLOQ) | | |
| DMSO | 10.0 | 3.3 | 9.8 | 91.4 |
| Cetuximab + DMSO | 7.6 | 0.49 | 1.5 | 4.4 |
| | | (<LLOQ) | | |
| Gefitinib | 4.1 | 0.68 | 1.7 | 4.7 |
| | | (<LLOQ) | | |
| Lapatinib | 1.4 | 0.56 | 1.4 | 3.3 |
| | | (<LLOQ) | | |
| iMEK AS703026 | 26.2 | 1.1 | 74.9 | 35.9 |

Per ug data is normalized from the ug sample concentration shown

### CONCLUSION

Combination of HER2 and EGFR inhibitors can increase the therapeutic index in cetuximab sensitive patients, due to inhibition of feedback mechanisms that are activated upon EGFR inhibition.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-20. (canceled)

21. A method for monitoring a subject receiving therapy with an EGFR inhibitor, the method comprising:
(a) detecting and/or quantifying the level of a complex in a sample taken from the subject at time (t₂), wherein the complex comprises an ErbB dimer, a HER3:PI3K complex, or a combination thereof; and
(b) comparing the level of the complex detected and/or quantified at (t₂) to the level of the complex detected and/or quantified at an earlier time (t₁); and
(c) determining whether to administer a combination therapy comprising the EGFR inhibitor with a HER2 inhibitor based upon a difference between the level of the complex at (t₂) compared to (t₁).

22. The method of claim 21, wherein the subject has colorectal cancer.

23. The method of claim 21, wherein the subject is sensitive to the EGFR inhibitor.

24. The method of claim 21, wherein the ErbB dimer is a HER2:HER3 heterodimer.

25. The method of claim 21, wherein step (a) comprises detecting and/or quantifying the level of the ErbB dimer and the level of the HER3:PI3K complex.

26. The method of claim 21, wherein the subject should be administered the combination therapy when the level of one or both of the complexes in the sample is higher at (t₂) compared to (t₁).

27. The method of claim 25, wherein the subject should be administered the combination therapy when the level of the ErbB dimer and the level of the HER3:PI3K complex in the sample are both higher at (t₂) compared to (t₁).

28. The method of claim 21, wherein (t₁) corresponds to a time before, or shortly after, initiation of treatment with the EGFR inhibitor.

29. The method of claim 21, wherein (t₁) corresponds to a time within about 0.5, 1, 2, 3, 4, 5, 6, 8, 12, 16, 20, or 24 hours after initiation of treatment with the EGFR inhibitor.

30. The method of claim 21, wherein (t₂) corresponds to a time between about 24 hours to about 12 months after initiation of treatment with the EGFR inhibitor.

31. The method of claim 21, wherein administration of the combination therapy reduces and/or inhibits the formation of one or both of the ErbB dimer and the HER3:PI3K complex.

32. The method of claim 21, wherein the method further comprises detecting and/or quantifying the expression level and/or activation level of HER2 and/or HER3 in the sample.

33. The method of claim 32, wherein the subject should be administered the combination therapy when the expression and/or activation level of HER2 and/or HER3 in the sample is higher at (t₂) compared to (t₁).

34. The method of claim 32, wherein administration of the combination therapy reduces and/or inhibits the level of expression and/or activation of HER2 and/or HER3.

35. The method of claim 21, wherein the EGFR inhibitor is selected from the group consisting of cetuximab (Erbitux®), gefitinib (Iressa®), erlotinib (Tarceva®), and a combination thereof.

36. The method of claim 21, wherein the HER2 inhibitor is selected from the group consisting of trastuzumab (Herceptin®), pertuzumab (2C4), and a combination thereof.

37. The method of claim 21, wherein the combination therapy comprises a dual EGFR/HER2 inhibitor such as lapatinib (Tykerb®).

38. The method of claim 21, wherein the sample is a cancer cell obtained from a tumor of the subject.

39. The method of claim 21, wherein the level of the complex is detected and/or quantified by a Collaborative Enzyme Enhanced Reactive Immunoassay (CEER).
